SEQUENCE LISTING (44 sequences)

SEQ ID NO: 1 - DNA, 1388 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtataggtcaataaaaagtagtcactggagcaacgatttccagtctctcttgatagtacatctcaccacatcagacctttaaaattttttgttgtcaaccatttactgttccttcttctccttccgtactgatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 2 - DNA, 1319 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtgtcccccagaaattccatataagcaatctaggaagttttggacctgggaaagctgtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 3 - DNA, 1302 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 4 - DNA, 1167 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 5 - DNA, 882 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 6 - DNA, 1164 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagatgctaatactggagaccgtggacagaccaataatgctgcttctgcatcagcttccaactccacctga

SEQ ID NO: 7 - DNA, 722 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctgcttcaacccccacaaatgccacagcagcgtcagatgctaatactggagaccgtggacagaccaataatgctgcttctgcatcagcttccaactccacctga

SEQ ID NO: 8 - DNA, 291 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaccgtggacagaccaataatgctgcttctgcatcagcttccaactccacctga

SEQ ID NO: 9 - DNA, 1263 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 10 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 11 - DNA, 723 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtataggtcaataa

SEQ ID NO: 12 - protein, 240 aa, Homo sapiens [sequence omitted]

SEQ ID NO: 13 - DNA, 837 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtgtcccccagaaattccatataa

SEQ ID NO: 14 - protein, 278 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVSPRNSI

SEQ ID NO: 15 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 16 - protein, 388 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 17 - protein, 293 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIELSSNPPLATILIPPHARIQAAASTPTNATAASDANTGDRGQTNNAASASASNST

SEQ ID NO: 18 - protein, 387 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQDANTGDRGQTNNAASASASNST

SEQ ID NO: 19 - protein, 240 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLLQPPQMPQQRQMLILETVDRPIMLLLHQLPTPP

SEQ ID NO: 20 - protein, 96 aa, Homo sapiens
MSGRPRTTSFAESCKPVQQPSAFGSMKVSRDKDGSKVTTVVATPGQGPDRPQEVSYTDTKVIGNGSFGVVYQAKLCDSGDRGQTNNAASASASNST

SEQ ID NO: 21 - DNA, 125 bp, Homo sapiens
gtcaataaaaagtagtcactggagcaacgatttccagtctctcttgatagtacatctcaccacatcagacctttaaaattttttgttgtcaaccatttactgttccttcttctccttccgtactg

SEQ ID NO: 22 - DNA, 56 bp, Homo sapiens
gtgtcccccagaaattccatataagcaatctaggaagttttggacctgggaaagct

SEQ ID NO: 23 - DNA, 39 bp, Homo sapiens
gattcgtcaggaacaggacatttcacctcaggagtgcgg

SEQ ID NO: 24 - DNA, 19 bp, Homo sapiens
gcaacgatttccagtctct

SEQ ID NO: 25 - DNA, 19 bp, Homo sapiens
gtagtcactggagcaacga

SEQ ID NO: 26 - DNA, 19 bp, Homo sapiens
ctcttgatagtacatctca

SEQ ID NO: 27 - DNA, 19 bp, Homo sapiens
tacatctcaccacatcaga

SEQ ID NO: 28 - DNA, 19 bp, Homo sapiens
tagtcactggagcaacgat

SEQ ID NO: 29 - DNA, 19 bp, Homo sapiens
aacgatttccagtctctct

SEQ ID NO: 30 - DNA, 19 bp, Homo sapiens
tctcaccacatcagacctt

SEQ ID NO: 31 - DNA, 19 bp, Homo sapiens
taagcaatctaggaagttt

SEQ ID NO: 32 - DNA, 19 bp, Homo sapiens
ttcgtcaggaacaggacat

SEQ ID NO: 33 - DNA, 19 bp, Homo sapiens
tcgtcaggaacaggacatt

SEQ ID NO: 34 - DNA, 19 bp, Homo sapiens
aggacatttcacctcagga

SEQ ID NO: 35 - DNA, 19 bp, Homo sapiens
ggacatttcacctcaggag

SEQ ID NO: 36 - DNA, 19 bp, Homo sapiens
atttcacctcaggagtgcg

SEQ ID NO: 37 - DNA, 30 bp, Artificial
  PCR Primer
catatgtcgacatgtcagggcggcccagaa

SEQ ID NO: 38 - DNA, 33 bp, Artificial
  PCR Primer
gatatgcggccgctcaggtggagttggaagctg

SEQ ID NO: 39 - DNA, 25 bp, Artificial
  PCR Primer
cagagttgatctttggagccactga

SEQ ID NO: 40 - DNA, 25 bp, Artificial
  PCR Primer
agaccatacatcagtacggaaggag

SEQ ID NO: 41 - DNA, 25 bp, Artificial
  PCR Primer
gctgagctgttactaggacaaccaa

SEQ ID NO: 42 - DNA, 23 bp, Artificial
  PCR Primer
gttcccaggacagctttcccagg

SEQ ID NO: 43 - DNA, 26 bp, Artificial
  PCR Primer
cagagaaatgaacccaaactacacag

SEQ ID NO: 44 - DNA, 23 bp, Artificial
  PCR Primer
ggtcggaagacccgcactcctga

(19) United States
(12) Patent Application Publication
(10) Pub. No.: US 2006/0127388 A1
(43) Pub. Date: Jun. 15, 2006

(54) Variants of glycogen synthase kinase 3 and uses thereof

(75) Inventors: Michael Cain, Stratham, NH (US); Paul J. Yaworsky, Rockland, MA (US)

Correspondence Address: KIRKPATRICK & LOCKHART NICHOLSON GRAHAM LLP/WYETH, STATE STREET FINANCIAL CENTER, ONE LINCOLN STREET, BOSTON, MA, 02111-2950, US

(73) Assignee: Wyeth, Madison, NJ (US)

(21) Appl. No.: 11/299,324

(22) Filed: Dec. 9, 2005

Related U.S. Application Data
(60) Provisional application No. 60/634,813, filed on Dec. 10, 2004.

(51) Int. Cl. A61K 38/45; C07H 21/04; C12P 21/06; C12N 9/12; C12N 5/08; C07K 16/40

(52) U.S. Cl. 424/94.5; 435/69.1; 435/194; 435/320.1; 435/366; 536/23.2; 530/388.26

(57) ABSTRACT

The present invention provides GSK3β variants, including splice variants expressed in bone or other tissues. These variants have a broad range of functional activity, from inhibiting the Wnt pathway to activating the pathway as dominant-negatives of the full-length GSK3β. These variants, as well as their modulators, can be used to treat diseases that involve GSK3β or its associated signaling pathways.

### REFERENCE TO RELATED APPLICATION

The present invention claims priority to, and the benefit of, U.S. application 60/634,813, filed Dec. 10, 2004, the complete disclosure of which is incorporated by reference herein for all purposes.

### TECHNICAL FIELD

The present invention relates to variants of glycogen synthase kinase 3 beta (GSK3β) and methods of using the same for the treatment of diseases that involve GSK3β or its associated signaling pathways.

### BACKGROUND

Glycogen synthase kinase 3 (GSK3) is a proline-directed serine/threonine protein kinase which can phosphorylate a wide array of proteins, such as metabolic enzymes, transcription factors, translation regulators, cell cycle factors, scaffold proteins, microtubule-associated proteins, and membrane proteins. At least two isoforms of GSK3 have been identified—namely, GSK3a and GSK3β. Both isoforms have similar kinase domains and are co-expressed in many tissues, but their cellular functions appear to be different. For instance, GSK3α is unable to functionally compensate for the loss of GSK3β in knock-out mice. In addition, GSK3β, not GSK3α, has been shown to play a major role in Wnt signaling.

GSK3 and its associated signaling pathways have been implicated in many diseases, such as osteoporosis, diabetes, Alzheimer's disease, obesity, bipolar disorder, atherosclerotic cardiovascular disease, essential hypertension, polycystic ovary syndrome, syndrome X, ischemia, traumatic brain injury, immunodeficiency and cancer. GSK3 has been linked to Type 2 diabetes through the insulin signaling pathway. Type 2 diabetes is characterized by increased insulin resistance in insulin responsive tissues. GSK3 is a negative regulator of the insulin signaling pathway. By phosphorylation, GSK3 inhibits glycogen synthase, resulting in elevated blood glucose levels. Insulin signaling deactivates GSK3, which in turn leads to more active glycogen synthase and increased glucose transport activity.

GSK3 has also been implicated in Alzheimer's disease. Alzheimer's disease is characterized by two main phenomena, neurofibrillary tangles within nerve cells and the deposition of amyloid plaques outside neurons. The neurofibrillary tangles are made up of fibrillar chains of hyperphosphorylated Tau. Tau normally promotes microtubule assembly and stabilization. However, the normal activity of Tau is significantly reduced by GSK3 hyperphosphorylation. Hyperphosphorylated Tau can accumulate around neurons to form neurofibrillary tangles. GSK3 has also been found in amyloid plaques and is suspected to play a central role in neuronal cell death.

GSK3 is also a key regulator in the Wnt signaling pathway. In the absence of Wnt signaling, GSK3 binds to Axin and APC to form a multi-protein complex. This complex can phosphorylate β-catenin, which is then targeted to the proteosome for degradation. When Wnt signals (e.g., Wnt1 or Wnt3a) are present, GSK3 disassociates from Axin, allowing accumulation of β-catenin in the cytoplasm. The accumulated β-catenin is translocated into the nucleus, interacting with TCF/LEF transcription factors to activate the transcription of Wnt target genes. This Wnt-mediated pathway is often referred to as the canonical Wnt pathway.

Wnt signaling has been demonstrated to be involved in bone density regulation. A point mutation in the Wnt receptor LRP5 has been linked to a high bone mass phenotype in humans (Little et al., (2002) Am. J. Hum. Genet., 70:11-19; and Boyden et al., (2002) N. Engl. J. Med., 346:1513-1521). In addition, Wnt pathway antagonists, such as DKK1 or SFRP1, have been showed to be involved in bone regulation. DKK1 can inhibit Wnt signaling by preventing the recruitment of Axin to the plasma membrane. In multiple myeloma patients, the expression of DKK1 in bone marrow plasma cells was directly correlated with focal bone lesions (Tian et al., (2003) N. Engl. J. Med., 349:2483-2494). DKK1 was also found to inhibit BMP-2 induced alkaline phosphatase expression (an early marker for bone) and the differentiation of osteoblast precursor cells in vitro. Furthermore, knock-out of another Wnt pathway antagonist, SFRP1, led to prolonged and enhanced trabecular bone density in adult animals. SFRP1 also prevented the apoptosis of osteoblast cells in vitro as well as in vivo. SFRP1 is believed to regulate bone mass by inhibiting Wnt-induced osteoblast maturation.

### SUMMARY OF THE INVENTION

The present invention provides numerous GSK3β variants with effects on the Wnt signaling pathway. In these variants, several amino acids (e.g. five or more consecutive amino acids) from one or more exons of GSK3-WT are absent as a result of, for example, a truncation of the protein, an internal deletion of at least five consecutive amino acids removing amino acids encoded by one or more exons or portions thereof, and/or replacement of at least five consecutive amino acids encoded by one or more exons or portions thereof. The absent amino acids correspond to amino acids encoded by one or more of GSK3-WT exons 2, 3, 4, 5, 6, 7, 8, 9, and 11. Other GSK3-WT amino acids may also be absent, although amino acids of exon 1 are preferably retained. In some embodiments, all or some amino acids (e.g. at least five consecutive amino acids) corresponding to GSK-3WT exon 10 are absent in addition to all or some amino acids corresponding to one or more of GSK3-WT exons 7, 8, and 9. Furthermore, one or more amino acids can be substituted to modulate biological activity (e.g. by substitution of GSK3 phosphorylation sites), to reduce immunogenicity (e.g. by substitution of predicted T-cell epitopes), or for other purposes, although it is preferred that no more than 10% of the amino acids (or, more preferably, no more than 5% of the amino acids) in the retained portion of GSK3-WT be substituted.

Among the GSK3β variants provided by the present invention are naturally-occurring splice variants that are expressed in bone or other tissues. These splice variants can be separated into three categories based on their activities in Wnt signaling. The first category includes GSK3Δ8 and GSK3-8b, which have the same or enhanced activity in the Wnt pathway as compared to the full-length GSK3β (GSK3-WT). The second category includes GSK3Δ7-9 and GSK3Δ10, which have less or similar activity in the Wnt pathway as compared to GSK3-WT. The third category includes GSK3-6b, GSK3-7b, GSK3Δ6-10 and GSK3Δ2-11, which have dominant-negative effects on the activity of the endogenous GSK3β protein, leading to significant augmentation in Wnt signaling.

The present invention features methods of using the dominant-negative variants of the present invention to treat diseases that involve GSK3β or its associated signaling pathways. Non-limiting examples of these diseases include osteoporosis, Type 2 diabetes, obesity, and Alzheimer's disease. Other bone, neurodegenerative or metabolic diseases can also be treated using the dominant-negative variants of the present invention. In many embodiments, the methods of the present invention include administering a pharmaceutical composition to a patient in need thereof, where the pharmaceutical composition comprises a dominant-negative variant of the present invention or an expression vector encoding the same. In one example, the dominant-negative variants employed in the present invention comprise amino acid residues encoded by exon 6b or exon 7b.

The present invention also features methods of using modulators of the GSK3β splice variants of the present invention to treat diseases that involve GSK3β or its associated signaling pathways. Modulators suitable for this purpose include, but are not limited to, antibodies, antisense molecules or RNAi sequences that are directed to GSK3Δ8 and GSK3-8b. In one embodiment, a GSK3Δ8 or GSK3-8b inhibitor is introduced or expressed in bone cells to treat osteoporosis or other bone disorders that are characterized by abnormal bone loss.

The present invention further features isolated polypeptides comprising the GSK3β variants of the present invention, or isolated polynucleotides encoding the same. In addition, the present invention features antibodies, antisense molecules, polynucleotide probes or primers, or RNAi sequences that are directed to the unique regions in the GSK3β splice variants. These unique regions include, for example, exon 6b for GSK3-6b, exon 7b for GSK3-7b, exon 8b for GSK3-8b, or the splicing junction regions of other splice variants.

Other features, objects, and advantages of the present invention are apparent in the detailed description that follows. It should be understood, however, that the detailed description, while indicating preferred embodiments of the present invention, is given by way of illustration only, not limitation. Various changes and modifications within the scope of the invention will become apparent to those skilled in the art from the detailed description

### BRIEF DESCRIPTION OF THE DRAWINGS

The drawings are provided for illustration, not limitation.

FIG. 1A schematically illustrates the nucleotide and protein structure of the full-length GSK3 gene (GSK3-WT). GSK3-WT is made up of 11 exons and encodes a protein having 3 distinct domains—namely, the N-terminal domain (amino acid residues 1-55), the kinase domain (amino acid residues 56-340), and the C-terminal domain (amino acid residues 341-421).

FIG. 1B schematically depicts the nucleotide structure of a GSK3β splice variant (GSK3-6b) which has a 125-bp insert between exon 6 and exon 7. The insertion introduces a stop codon, resulting in a truncated protein with 240 amino acid residues.

FIG. 1C schematically describes the nucleotide structure of another GSK3β splice variant (GSK3-7b) which has a 56-bp insert between exon 7 and exon 8. The insertion also introduces a stop codon, creating a truncated protein with 278 amino acid residues.

FIG. 1D schematically shows the nucleotide structure of yet another GSK3β splice variant (GSK3-8b) which has a 39-bp insert between exon 8 and exon 9. The insertion does not change the reading frame, but does increase the protein size to 433 amino acid residues.

FIG. 1E schematically depicts the nucleotide structure of another GSK3β splice variant (GSK3Δ8) which misses exon 8. The variant retains the original open-reading frame (ORF) and encodes a protein of 388 amino acid residues.

FIG. 1F is the nucleotide structure of still another GSK3β splice variant (GSK3Δ7-9) which misses exons 7, 8 and 9. The variant retains the original ORF and encodes a protein of 293 amino acid residues.

FIG. 1G shows the nucleotide structure of another GSK3β splice variant (GSK3Δ10), which misses exon 10. The variant retains the ORF and encodes a protein of 387 amino acid residues.

FIG. 1H schematically demonstrates the nucleotide structure of a GSK3β splice variant (GSK3Δ6-10) which includes a 541-bp deletion encompassing most of exon 6 and all of exons 7-10. The 541-bp deletion causes a shift in the reading frame which extends the ORF beyond exon 11, resulting in a protein with over 240 amino acid residues.

FIG. 1I shows the nucleotide structure of another GSK3β splice variant (GSK3Δ2-11) which has a 972-bp deletion extending from exon 2 to exon 11. The variant retains the original ORF and encodes a protein having 96 amino acid residues.

FIGS. 2A, 2B and 2C illustrate the sequences and locations of the insertions in GSK3-6b, GSK3-7b and GSK3-8b, respectively. The location of each insertion (measured by the amino acid sequence of GSK3-WT) is: GSK3-6b, after amino acid 238; GSK3-7b, after amino acid 271; and GSK3-8b, after amino acid 303.

FIG. 3 compares a known consensus sequence for intron splicing (Schaffer et al., GENE, 302:73-81 (2003)) to the sequences immediately adjacent to the deleted regions in GSK3Δ8, GSK3Δ7-9, GSK3Δ10, GSK3Δ6-10, and GSK3Δ2-11, respectively.

FIG. 4 is a multiple tissue Northern blot for GSK3-WT. Two major bands were observed at 8.3 kb and 2.8 kb (top two arrows in lane 3). Two additional bands at about 1.0 kb and 2.0 kb (bottom two arrows in lane 3) were also detected in at least heart, skeletal muscle and liver.

FIG. 5 is a multiple tissue Northern blot for GSK3-8b. Two major bands were seen at about 4.5 kb and 2.2 kb (arrows in lane 3). Two additional bands at about 1.0 kb and 0.5 kb (arrows in lane 8) were also detected in at least heart, skeletal muscle and liver.

FIG. 6 illustrates a multiple tissue Northern blot for GSK3-6b. Overnight exposure of the Northern blot indicated a major band at about 1.8 kb in skeletal muscle (arrow).

FIG. 7 shows overnight exposure of a multiple tissue Northern blot for GSK3-7b. Multiple bands were observed in heart, skeletal muscle, kidney, liver, small intestine, and placenta. Bands at about 1.6 kb and 1.4 kb were detected in skeletal muscle, together with an additional band at about 8.0 kb.

FIG. 8 describes RT-PCR results for control gene GAPDH in different bone cell lines (U2OS, TE85, SAOS2, and MG63). The predicted size of the RT-PCR product was about 100 bp. “No-RT” controls were run for each different RNA source. 100 ng of cDNA was used for each reaction. All cell lines expressed GAPDH, and intensities of the bands indicate greater or lesser amounts of RNA used per RT-PCR reaction as well as the relative abundance of GAPDH expressed in each cell line.

FIG. 9 shows RT-PCR results for GSK3-WT in different bone cell lines (U2OS, TE85, SAOS2, and MG63). “No-RT” controls were run for each different RNA source. 100 ng of cloned GSK3-WT cDNA was used as a positive control (lane 12). The expected PCR product size was about 1,300 bp. Expression of GSK3-WT was detected in U2OS, TE85 and SAOS2.

FIGS. 10A, 10B and 10C illustrate RT-PCR results for GSK3-6b, GSK3-7b and GSK3-8b, respectively, in different bone cell lines (U2OS, TE85, SAOS2, and MG63). 100 ng of total RNA was used for each reaction. 100 ng of cloned variant cDNA was used as positive controls (lanes 12). (A) GSK3-6b was detected in U2OS, TE85 and liver. The expected band size was about 200 bp. (B) GSK3-7b was detected in U2OS, SAOS2, and MG63. The expected band size was about 100 bp. (C) GSK3-8b was detectable in U2OS, TE85, SAOS2, MG63, and liver. The expected band size was about 100 bp. Arrows indicate the PCR product which was subcloned and confirmed by sequencing.

FIG. 11 demonstrates the activation of the Wnt pathway by Wnt3a. Two replicate experiments are shown where empty vector, along with reporter constructs, was transfected into U2OS cells. In both experiments, about 3-fold increase in activation of the Wnt pathway was observed when the cells were treated with Wnt3a conditioned medium (+Wnt3a) over the untreated cells (−Wnt3a). * indicates statistically significant difference between Wnt3a-treated cells and untreated cells (p-value<0.05).

FIG. 12 shows GSK3-WT inhibition of the Wnt pathway. Two replicate experiments are shown where GSK3-WT, along with reporter constructs, was transfected into U2OS cells. An 18% and a 24% inhibition of the Wnt pathway, as compared to empty vector, were observed in untreated cells. Cells that were treated with Wnt3a showed a 32% and a 41% inhibition. Dotted line indicates empty vector control which was set equal to one. * indicates statistically significant difference from empty vector control (p-value<0.05).

FIG. 13 illustrates TCF-luciferase assay results for GSK3β splice variants using untreated U2OS cells. Each GSK3β variant, along with reporter constructs, was transfected into U2OS cells untreated with Wnt3a. Data was normalized to GSK3-WT. No significant differences were found among the variants investigated as compared to GSK3-WT. Replicate experiments were performed for each variant (Expt#1 and Expt#2).

FIG. 14 describes TCF-luciferase assay results for GSK3β splice variants using Wnt3a-treated U2OS cells. The variants can be separated into three categories based on the assay results. The first category includes variants that had activities equal to or better than GSK3-WT. GSK3Δ8 had nearly identical activity as GSK3-WT, while GSK3-8b showed improved activity (by 30% and 48%) in inhibiting the Wnt pathway. The second category includes variants that appeared to be as effective as or less effective than GSK3-WT in inhibiting the Wnt pathway. GSK3Δ7-9 had about 1.4-1.5 fold increase in the Wnt pathway activity, while GSK3Δ10 had about 1.5-1.9 fold increase in the activity. A third category includes variants that caused significant increases in luciferase activity as compared to GSK3-WT. The increases in luciferase activity were: GSK3Δ6-10 (about 2.0-2.1 fold), GSK3Δ2-11 (about 1.9-2.9 fold), GSK3-6b (about 2.0-2.8 fold), and GSK3-8b (about 2.3-3.0 fold). These increases were statistically significant with p-values<0.05. Replicate experiments were performed for each variant (Expt#1 and Expt#2).

### DETAILED DESCRIPTION

The present invention provides numerous GSK3β variants with effects on the Wnt signaling pathway. In these variants, several amino acids (e.g. five or more consecutive amino acids) from one or more exons of GSK3-WT are absent as a result of, for example, a truncation of the protein, an internal deletion of at least five consecutive amino acids removing amino acids encoded by one or more exons or portions thereof, and/or replacement of at least five consecutive amino acids encoded by one or more exons or portions thereof. The absent amino acids correspond to amino acids encoded by one or more of GSK3-WT exons 2, 3, 4, 5, 6, 7, 8, 9, and 11. Other GSK3-WT amino acids may also be absent, although amino acids of exon 1 are preferably retained. In some embodiments, all or some amino acids (e.g. at least five consecutive amino acids) corresponding to GSK-3WT exon 10 are absent in addition to all or some amino acids corresponding to one or more of GSK3-WT exons 7, 8, and 9. Furthermore, one or more amino acids can be substituted to modulate biological activity (e.g. by substitution of GSK3 phosphorylation sites), to reduce immunogenicity (e.g. by substitution of predicted T-cell epitopes), or for other purposes, although it is preferred that no more than 10% of the amino acids (or, more preferably, no more than 5% of the amino acids) in the retained portion of GSK3-WT be substituted.

Among the GSK3β variants provided by the present invention are naturally-occurring splice variants that are expressed in human bone or other tissues. These splice variants include GSK3-6b (SEQ ID NO:1), GSK3-7b (SEQ ID NO:2), GSK3-8b (SEQ ID NO:3), GSK3Δ8 (SEQ ID NO:4), GSK3Δ7-9 (SEQ ID NO:5), GSK3Δ10 (SEQ ID NO:6), GSK3Δ6-10 (SEQ ID NO:7), and GSK3Δ2-11 (SEQ ID NO:8). These splice variants can be separated into three groups based on their activities in inhibiting the Wnt signaling pathway. The first group includes GSK3Δ8 and GSK3-8b. The proteins encoded by these two variants have Wnt pathway activities equal to or better than the full-length GSK3β protein (GSK3-WT). The second group includes GSK3Δ7-9 and GSK3Δ10, which encode proteins that have less or similar activity in Wnt signaling than GSK3-WT. The third group includes GSK3Δ6-10, GSK3Δ2-11, GSK3-6b, and GSK3-7b, which are dominant-negatives of the endogenous full-length GSK3β protein. The splice variants identified by the present invention may represent a complex yet specific mechanism for signal transduction regulation in human cells.

The dominant-negative variants of the present invention can be used to inhibit GSK3β activities in human cells or tissues. The variants of the present invention can also be used to prepare or identify GSK3β modulators, such as antibodies, antisense molecules or RNAi sequences. These modulators, as well as the dominant-negative variants, can be used to treat diseases that are associated with GSK3β or its related signal transduction pathways (e.g., the Wnt pathway). Non-limiting examples of diseases that are amenable to the present invention include bone diseases (e.g., osteoporosis), neurodegenerative diseases (e.g., Alzheimer's disease), and metabolic diseases (e.g., Type 2 diabetes).

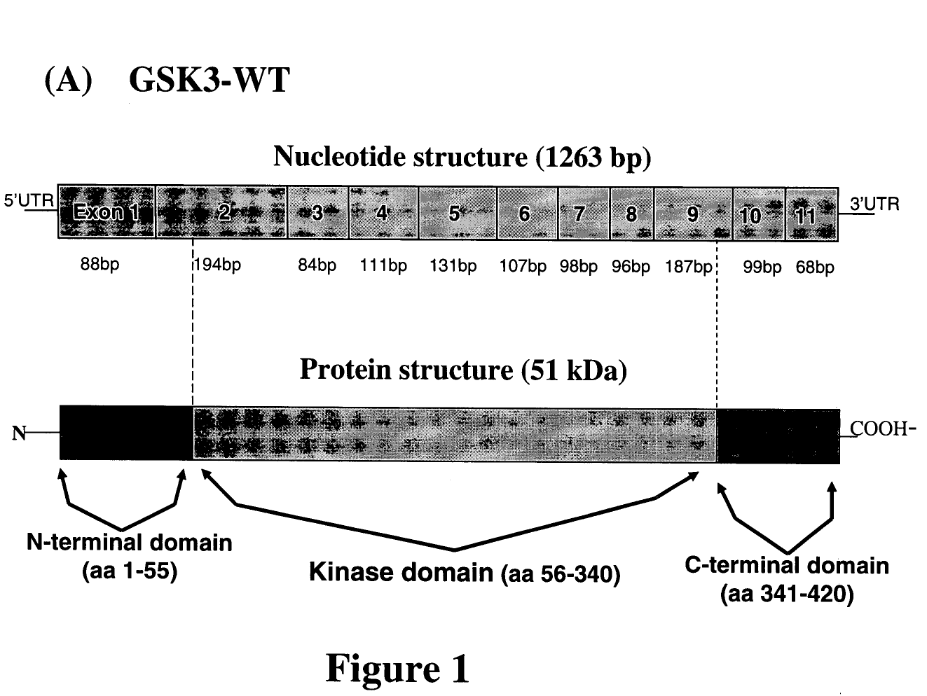

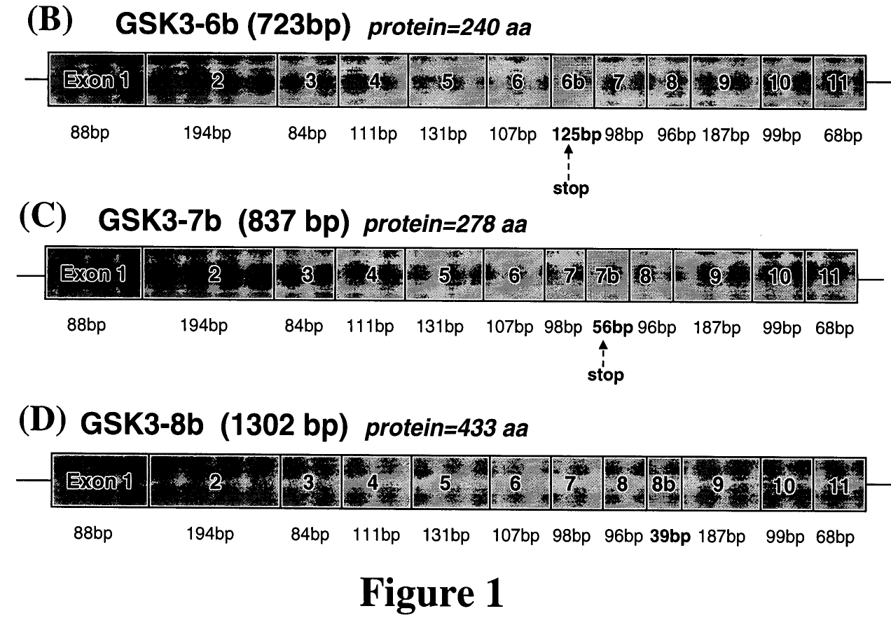

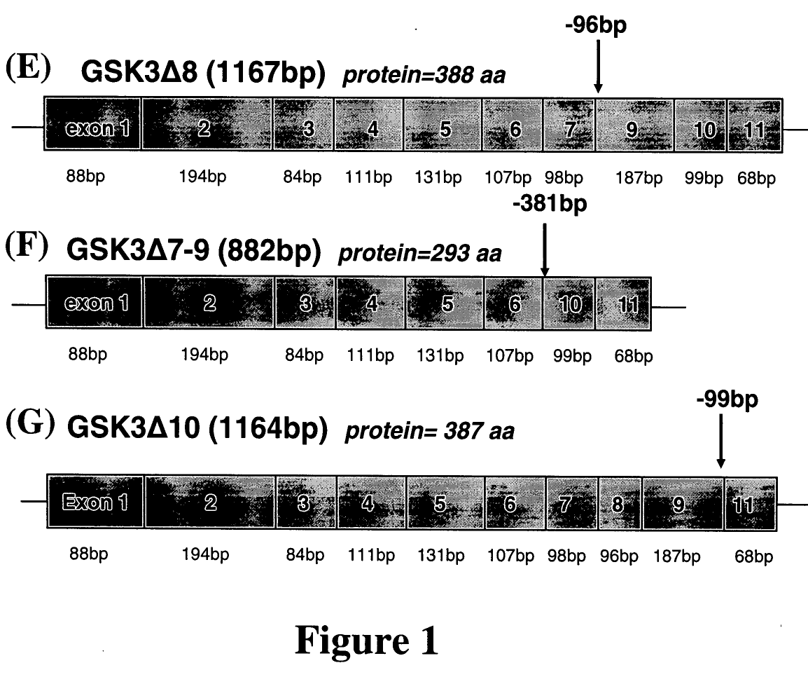

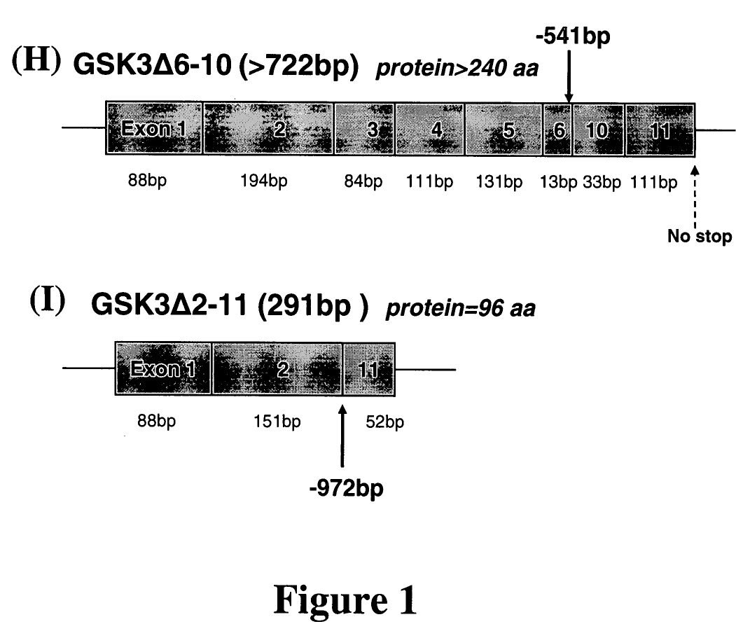

The full-length GSK3β gene (GSK3-WT) has 11 exons and encodes a protein with three distinct domains: an N-terminal domain (amino acids 1-55), a kinase domain (amino acids 56-340) and a C-terminal domain (amino acids 341-420) (FIG. 1A). The open reading frame (ORF) of GSK3-WT is depicted in SEQ ID NO:9, and the encoded amino acid sequence is provided in SEQ ID NO:10.

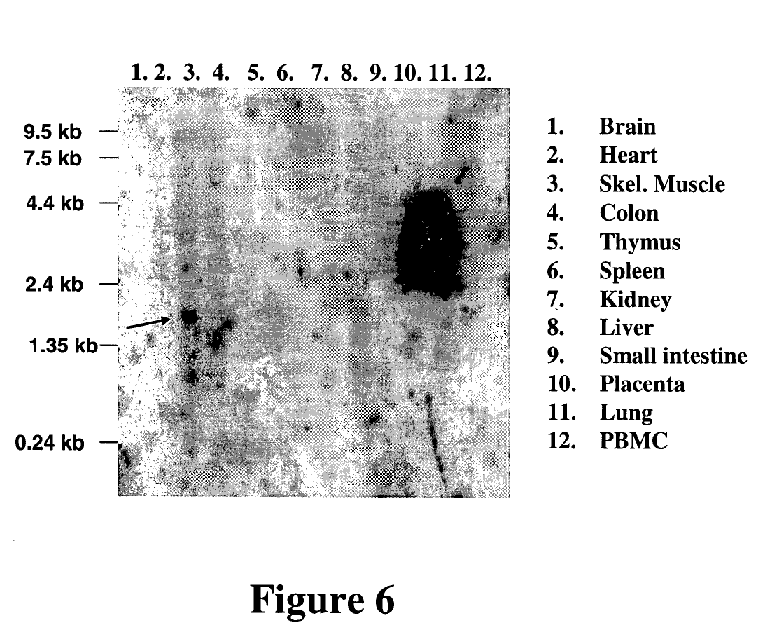

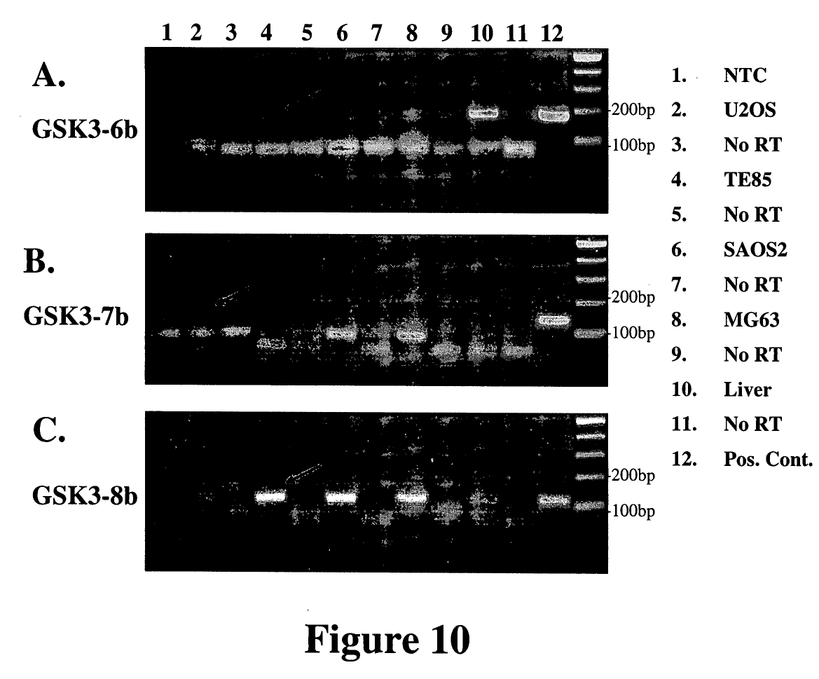

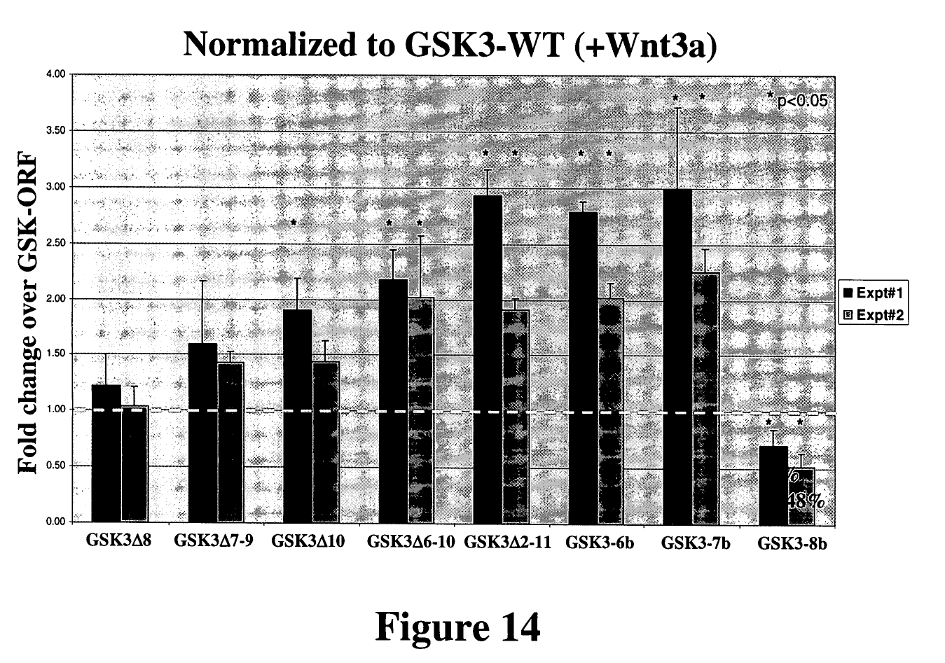

GSK3-6b includes a 125-bp insertion between exon 6 and exon 7 (FIG. 1B). This insertion is referred to as exon 6b. The insertion of exon 6b causes a shift in the GSK6β reading frame, introducing a stop codon at amino acid 241. The nucleotide sequence of GSK3-6b is depicted in SEQ ID NO:1. The ORF of GSK3-6b (consisting of all of the codons from the start codon to the stop codon without introns or other intervening sequences) is depicted in SEQ ID NO:11, and the encoded polypeptide is depicted in SEQ ID NO:12. As compared to GSK3-WT, GSK3-6b exhibited about 2.0-2.8 fold increase in the reporter gene activity in TCF-luciferase assay (FIG. 14 and Example 4). TCF-luciferase assay measures the activation of the Wnt pathway, as TCF transcription factors are known downstream binding partners of β-catenin. A significant increase in the reporter gene activity indicates that GSK3-6b has a dominant-negative effect on the activity of the endogenous full-length GSK3β protein in Wnt signaling. Expression of GSK3-6b was observed in bone cells (FIG. 10A) and skeletal muscle (FIG. 6, lane 3), suggesting its involvement in bone regulation and glycogen metabolism in muscles.

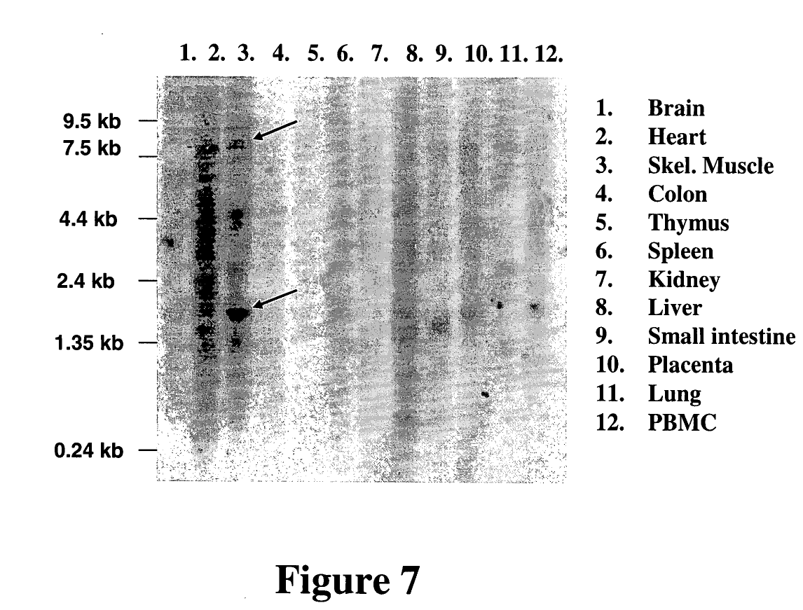

GSK3-7b includes a 56-bp insertion between exon 7 and exon 8 (FIG. 1C). This insertion is referred to as exon 7b. Like exon 6b, exon 7b also introduces a stop codon in the GSK3β reading frame, resulting in an ORF that encodes 278 amino acid residues. The nucleotide sequence of GSK3-7b is depicted in SEQ ID NO:2. The ORF sequence (from the start codon to the stop codon without introns or other intervening sequences) is depicted in SEQ ID NO:13, and the encoded protein is described in SEQ ID NO:14. Like GSK3-6b, GSK3-7b is a dominant-negative of the endogenous full-length GSK3β (FIG. 14 and Example 4). Dominant-negatives are altered forms of a protein that lack the normal functional activity. Over-expression of dominant-negatives can out-compete the endogenous protein by, for example, binding to or sequestering its interaction partners. This effectively inhibits the cellular function of the endogenous protein. GSK3-7b is expressed in bone cells (FIG. 10B) and skeletal muscle (FIG. 7), suggesting its involvement in bone regulation and glycogen metabolism in muscles. GSK3-7b is also expressed in heart, liver, small intestine, and placenta (FIG. 7).

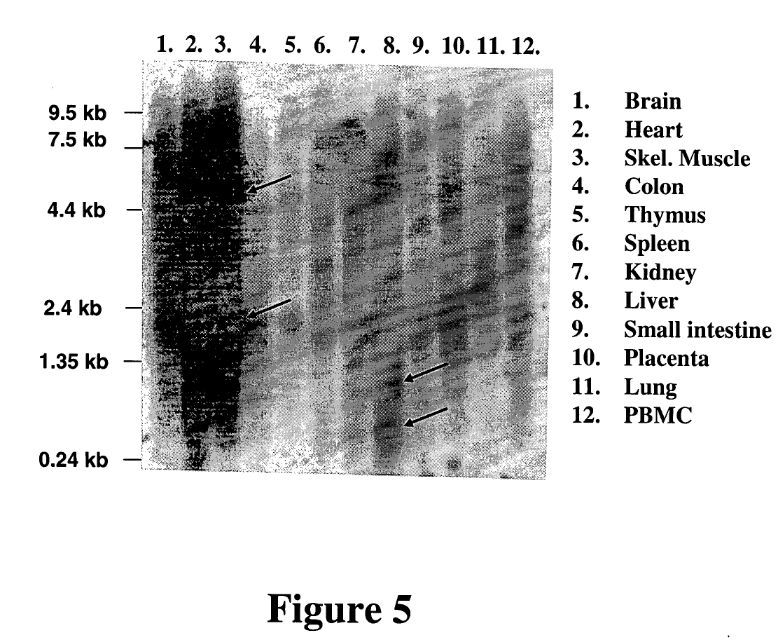

GSK3-8b includes a 39-bp insertion between exon 8 and exon 9 (FIG. 1D). Despite the insertion, GSK3-8b retains the original open reading frame but adds 13 additional amino acids after amino acid residue 303. The ORF of GSK3-8b is depicted in SEQ ID NO:3, and the encoded polypeptide in SEQ ID NO:15. RT-PCR and Northern analysis demonstrated that GSK3-8b is expressed in a variety of tissues, including but not limited to, bone, brain, heart, skeletal muscle, thymus, spleen, kidney, liver, small intestine, placenta, lung, and peripheral blood mononuclear cells (FIGS. 5 and 10C). The fact that GSK3-8b is expressed in metabolic tissues, such as liver and muscle, indicates its involvement in glycogen metabolism or insulin signaling. In addition, as demonstrated in Example 4, GSK3-8b possesses an enhanced activity in the regulation of the Wnt pathway as compared to GSK3-WT (FIG. 14). All of these data suggest that GSK3-8b is a major drug target for the treatment of diseases that involve GSK3β or its associated signaling pathways. GSK3-8b was described in Schaffer et al., (2003) Gene, 302:73-81 and Mukai et al., (2002) J. Neurochemistry, 81:1073-1083, and has been thought to be primarily expressed in brain.

GSK3Δ8 (SEQ ID NO:4) lacks exon 8 (FIG. 1E) and encodes a protein having 388 amino acid residues, the sequence of which is depicted in SEQ ID NO:16. TCF-luciferase assay indicated that GSK3Δ8 has nearly the same activity as the full-length GSK3β protein, suggesting that the complete loss of exon 8 does not affect GSK3β activity in the regulation of the Wnt pathway.

GSK3Δ7-9 (SEQ ID NO:5) and GSK3Δ10 (SEQ ID NO:6) lack exons 7-9 and exon 10, respectively (FIGS. 1F and G). Both splice variants retain the original GSK3β reading frame. The amino acid sequences encoded by these two variants are depicted in SEQ ID NOs: 17 and 18, respectively. TCF-luciferase assays demonstrated that both splice variants have less activity in the regulation of the Wnt pathway as compared to GSK3-WT (FIG. 14). GSK3Δ10 was also described in Schaffer et al., (2003) Gene, 302:73-81.

GSK3Δ6-10 includes a 541-bp deletion encompassing the most part of exon 6, the entire exons 7-9, and the majority portion of exon 10 (FIG. 1H). The deletion introduces a shift in the reading frame, which potentially extends the length of the ORF beyond exon 11. A partial ORF sequence of GSK3Δ6-10 is depicted in SEQ ID NO:7, and the encoded amino acid sequence is provided in SEQ ID NO:19. Like GSK3-6b and GSK3-7b, GSK3Δ6-10 is a dominant-negative of the endogenous full-length GSK3β, and can produce about 2.0-2.1 fold increase in the reporter gene activity in TCF-luciferase assay as compared to GSK3-WT (FIG. 14 and Example 4).

GSK3Δ2-11 (SEQ ID NO:8) includes a 972-bp deletion extending from exon 2 to exon 11 (FIG. 1I). The deletion, however, does not alter the original GSK3β reading frame. The polypeptide sequence encoded by GSK3Δ2-11 is depicted in SEQ ID NO:20. GSK3Δ2-11 also has the dominant-negative effect on the activity of the endogenous full-length GSK3β protein. TCF-luciferase assay showed that GSK3Δ2-11 produced about 1.9-2.9 fold increase in the reporter gene activity as compared to GSK3-WT.

GSK3Δ2-11 and GSK3Δ6-10 are both missing large portions of the GSK3 kinase domain and, therefore, may function as “kinase dead” dominant-negatives. These variants can out-compete the endogenous protein, reducing the inhibitory effect of the endogenous protein on the Wnt pathway.

The dominant-negative variants of the present invention can be used to inhibit GSK3β activities in human cells or tissues. Inhibition of GSK3β activities is desirable for treating many diseases that involve GSK3β or its associated signal transduction pathways. Examples of these diseases include, but are not limited to, osteoporosis, diabetes, obesity, Alzheimer's disease, atherosclerotic cardiovascular disease, essential hypertension, polycystic ovary syndrome, syndrome X, ischemia, traumatic brain injury, bipolar disorder, immunodeficiency, and cancer.

Osteoporosis is characterized by progressive loss of bone architecture and mineralization leading to the loss in bone strength and an increased fracture rate. The skeleton is constantly being remodeled by a balance between osteoblasts that lay down new bone and osteoclasts that breakdown, or resorb, bone. In some disease conditions or advancing age, the balance between bone formation and resorption is disrupted, and bone is removed at a faster rate. Such a prolonged imbalance of resorption over formation leads to weaker bone structure and a higher risk of fractures. Most drugs used today for the treatment of osteoporosis are anti-resorptive drugs, which help reduce the rate of bone loss.

The present application features the use of GSK3β dominant-negatives (e.g., GSK3-6b, GSK3-7b, GSK3Δ6-10, or GSK3Δ2-11) for treating or preventing osteoporosis. Introducing GSK3β dominant-negatives into bone cells (e.g., osteoblasts or their precursor cells) inhibits the activity of the endogenous full-length GSK3β, thereby activating the Wnt pathway and increasing the rate of bone formation. This is similar to the action of lipoprotein receptor-related protein 5 (LRP5) with gain of function mutations (Little et al., (2002) Am. J. Hum. Genet., 70:11-19). LRP5 functions as a Wnt co-receptor in the osteoblast signaling pathway, and gain-of-function mutations in LRP5 increase Wnt signaling by impairing the action of normal antagonists of the Wnt pathway, resulting in a high bone mass phenotype.

For the same reason, GSK3β dominant-negatives can be used for treating other bone diseases that are associated with increased bone resorption or reduced bone formation. These diseases include, but are not limited to, osteopenia, osteoarthritis, rheumatoid arthritis, periprosthetic bone loss, osteolysis, metastatic bone disease, and Paget's disease. Other metabolic bone disorders or arthritic conditions can also be treated using the GSK3β dominant-negatives of the present invention.

The present invention also features the use of GSK3β dominant-negatives for treating or preventing Alzheimer's disease. GSK3β has been implicated in the biological pathways relating to Alzheimer's disease. The characteristic pathological features of Alzheimer's disease are extracellular plaques of an abnormally processed form of the amyloid precursor protein (APP), so called β-amyloid peptide and the development of intracellular neurofibrillary tangles containing paired helical filaments (PHF) that consist largely of hyperphosphorylated Tau protein. GSK3 can phosphorylate Tau protein in vitro on the abnormal sites that are characteristic of PHF Tau. Furthermore, the GSK3 kinase inhibitor, lithium chloride, can block Tau hyperphosphorylation in cells. In addition, GSK3β can bind to another key protein involved in the pathogenesis of Alzheimer's disease, presenilin 1 (PS1). Mutations in the PS1 gene lead to increased production of β-AP, and the mutant PS1 proteins bind more tightly to GSK3β and potentiate the phosphorylation of Tau. Moreover, increased association of GSK3β with mutant PS1 may account for the reduced levels of β-catenin that have been observed in the brains of PS1-mutant AD patients. Consistent with these observations, it has been shown that injection of GSK3 antisense but not sense, blocks the pathological effects of β-AP on neurons in vitro, resulting in delay in the onset of cell death and increased cell survival. Like other GSK3β inhibitors, GSK3β dominant-negatives can be used to inhibit the activity of the endogenous GSK3β in neuronal cells, thereby slowing or reversing the progression of Alzheimer's disease.

GSK3β dominant-negatives can also be used to treat or prevent other neurodegenerative diseases or nervous system disorders that involve abnormal activities in GSK3β or its associated signaling pathways. These diseases or disorders include, but are not limited to, bipolar disorder (manic depressive syndrome), Huntington's disease, Parkinson's disease, AIDS-associated dementia, amyotrophic lateral sclerosis and multiple sclerosis. One mechanism by which GSK3β dominant-negatives can act to treat these diseases is to increase the survival of neurons subjected to aberrantly high levels of excitation induced by the neurotransmitter, glutamate. Glutamate-induced neuronal excitotoxicity is believed to be a major cause of neurodegeneration associated with acute damage, such as in cerebral ischemia, traumatic brain injury, and bacterial infection. Excessive glutamate signaling is also believed to be a factor in the chronic neuronal damage seen in diseases such as Alzheimer's, Huntington's, Parkinson's, AIDS-associated dementia, amyotrophic lateral sclerosis and multiple sclerosis. Wnt signaling has been reported to be able to protect against apoptosis in cells exposed to chemical stress. Consequently, GSK3β dominant-negatives can be used to activate the Wnt signaling pathway in neurons or glial cells, improving their survival against glutamate-induced neuronal excitotoxicity.

In addition, the present invention features the use of GSK3β dominant-negatives for treating or preventing diabetes. Type 2 diabetes is initially characterized by decreased sensitivity to insulin and a compensatory elevation in circulating insulin concentrations, the latter of which is required to maintain normal blood glucose levels. Increased insulin levels are caused by increased secretion from the pancreatic beta cells, and the resulting hyperinsulinemia is associated with cardiovascular complications of diabetes. As insulin resistance worsens, the demand on the pancreatic beta cells steadily increases until the pancreas can no longer provide adequate levels of insulin, resulting in elevated levels of glucose in the blood. Ultimately, overt hyperglycemia and hyperlipidemia occur, leading to the devastating long-term complications associated with diabetes, including cardiovascular disease, renal failure and blindness.

GSK3 inhibition can stimulate insulin-dependent processes and is, therefore, useful for the treatment of Type 2 diabetes. Recent data obtained by using lithium salts as GSK3 inhibitors supports this notion. The lithium ion has been reported to have antidiabetic effects including the ability to reduce plasma glucose levels, increase glycogen uptake, potentiate insulin, up-regulate glucose synthase activity and to stimulate glycogen synthesis in skin, muscle and fat cells. GSK3β dominant-negatives can be similarly used to inhibit GSK3β activities in metabolic tissues, such as liver or skeletal muscle, thereby preventing or slowing the progression of Type 2 diabetes. Likewise, GSK3β dominant-negatives can be used to reduce fat accumulation in adipose or metabolic tissues for the treatment or prevention of obesity.

In addition to GSK3-6b, GSK3-7b, GSK3Δ6-10, or GSK3Δ2-11, the present invention contemplates the use of other dominant-negatives for inhibiting the activities of the endogenous GSK3β. In many embodiments, these dominant-negatives are encoded by modified GSK3β genes that lack one or more exons. Deletion of an exon can be either partial or complete. In one example, a dominant-negative of the present invention comprises or consists essentially of a polypeptide encoded by exon 1 and part of exon 2 of GSK3-WT (e.g., amino acids 1-60, amino acids 1-75, amino acids 1-80, amino acids 1-85, or amino acids 1-90 of SEQ ID NO:10). In another example, a dominant-negative of the present invention comprises or consisting essentially of a polypeptide encoded by exons 1 and 2 and one or more exons selected from exons 3, 4, 5 and 6 of GSK3-WT. In still another example, a dominant-negative of the present invention includes or consists essentially of a polypeptide encoded by exon 6b or 7b and/or sequences adjacent thereto (e.g., amino acids 239-240, 235-240, or 230-240 of SEQ ID NO:12 (GSK3-6b), or amino acids 273-278, 264-278, or 259-278 of SEQ ID NO:14 (GSK3-7b)). The dominant-negatives of the present invention can also be prepared by introducing stop codons into GSK3β coding sequences (such as exons 3, 4, 5, 6 or 7), creating truncations within the kinase domain. Exemplary insertions include, but are not limited to, exon 6b or exon 7b. Other amino acid substitutions, deletions, insertions, or modifications can also be used to generate GSK3β dominant-negatives.

The present invention further features modulators of the GSK3β splice variants of the present invention. Like GSK3β dominant-negatives, these modulators can be used to modify the activities of the endogenous GSK3β proteins for the treatment or prevention of diseases. For instance, GSK3-6b is a dominant-negative of the endogenous full-length GSK3β protein. Therefore, a GSK3-6b enhancer can be used to increase its dominant effect and, therefore, improve GSK3-6b's therapeutic value. Conversely, a GSK3-6b inhibitor can be used to reduce the dominant-negative effect of GSK3-6b, restoring the activity of the endogenous full-length GSK3β protein and thereby inactivating the Wnt pathway. Excessive activity in Wnt signaling has been implicated in cancer (Van de Wetering et al., CELL, 111:241-50 (2002)). Thus, a GSK3-6b inhibitor can be used to treat these conditions by suppressing the Wnt pathway.

Likewise, GSK3-7b enhancers can be used to increase the dominant effect of GSK3-7b on the activity of the endogenous GSK3-WT, while GSK3-7b inhibitors can be used to treat diseases associated with abnormally high activities in Wnt signaling or abnormally low activities in GSK3β.

As compared to GSK3-WT, GSK3-8b has an enhanced activity in the regulation of the Wnt pathway. GSK3-8b is expressed in a variety of tissues, including bone, brain, heart, skeletal muscle, thymus, spleen, kidney, liver, small intestine, placenta, lung, and peripheral blood mononuclear cells. Therefore, GSK3-8b inhibitors can be used to activate the Wnt pathway in these tissues by suppressing the activity of GSK3-8b. Activating Wnt signaling, via GSK inhibition, can result in a beneficial increase in bone mass (Little et al., (2002) Am. J. Hum. Genet., 70:11-19). In addition, GSK3 inhibition can generate anabolic bone responses (Zhao et al., (2003) J. Bone and Mineral Res. Annual Meeting, 18(2), S29, 1108). Modulators of other GSK3β splice variants are also contemplated by the present invention.

GSK3β modulators can be of any type of molecule, such as peptide, small molecule, or chemical compound. In many cases, a GSK3β modulator can enhance or inhibit (e.g., reduce or eliminate) the functionality of a corresponding GSK3β splice variant by at least 20%, 30%, 40%, 50%, 60%, 70%, 80%, 90% or more. In many other cases, a GSK3β modulator can specifically enhance or inhibit the functionality of a particular GSK3β splice variant. By “specifically,” it means that the modulator can modify the functionality of the corresponding variant, but not others. Identification of a GSK3β modulator typically includes detecting a biological function of a GSK3β splice variant in the presence or absence of a candidate molecule, where a change in the biological function in the presence of the candidate molecule, as compared to that in the absence of the molecule, suggests that the candidate molecule is a modulator of the GSK3β splice variant. Any assay format can be used for this purpose, such as cell-based assays, array-based assays, or standard biochemical assays. In one embodiment, the biological function (e.g., kinase activity or dominant-negative effect) of a GSK3β splice variant is evaluated using the TCF-luciferase assay described in Examples 1 and 4. Compound libraries, phage display techniques, or other high throughput screening methods can also be used.

In addition, GSK3β modulators can be identified using three-dimensional structural analysis or computer aided drug design. The latter method may entail determination of binding sites for modulators based on the three-dimensional structures of GSK3β splice variants and their binding partners (e.g., Axin, FRAT or GSK3β substrates). Molecules reactive with the binding site(s) on a GSK3β splice variant or its substrate(s) are selected. Candidate molecules are then assayed for determining any modulation effect.

The present invention further features antibodies against GSK3β splice variants. These antibodies can be used to modulate the activities of the corresponding GSK3β splice variants. These antibodies can also be used for the detection or isolation of GSK3β splice variants. Exemplary antibodies suitable for the present invention include, but are not limited to, polyclonal, monoclonal, mono-specific, poly-specific, non-specific, humanized, human, single-chain, chimeric, synthetic, recombinant, hybrid, mutated, grafted, or in vitro generated antibodies. The antibodies of the present invention can also be Fab, F(ab′)₂, Fv, scFv, Fd, dAb, or other antibody fragments that retain the antigen-binding function. In many instances, an antibody of the present invention can bind to a corresponding GSK3β splice variant with an affinity constant of at least 10⁴ M⁻¹, 10⁵ M⁻¹, 10⁶ M⁻¹, 10⁷ M⁻¹, or stronger.

In many embodiments, an antibody of the present invention specifically recognizes a particular GSK3β splice variant. A variant-specific antibody can be prepared by using the epitopes located in the unique region of each corresponding splice variant. These unique regions include: for GSK3-6b, amino acids 239-240 of SEQ ID NO:12 which are encoded by exon 6b; for GSK3-7b, amino acids 273-278 of SEQ ID NO:14 which are encoded by exon 7b; for GSK3-8b, amino acids 304-316 of SEQ ID NO:15 which are encoded by exon 8b; for GSK3Δ8, amino acids 270-273 of SEQ ID NO:16 which are encoded by the junction between exon 7 and exon 9; for GSK3Δ7-9, amino acids 237-240 of SEQ ID NO:17 which are encoded by the junction between exon 6 and exon 10; for GSK3Δ10, amino acids 364-367 of SEQ ID NO:18 which are encoded by the junction between exon 9 and exon 11; for GSK3Δ6-10, amino acids 208-240 of SEQ ID NO:19 which are encoded by the shifted ORF after the 541-bp deletion; and for GSK3Δ2-11, amino acids 77-81 of SEQ ID NO:20 which are encoded by the junction between the remaining exon 2 and the remaining exon 11.

Antibodies can be raised against these unique regions by using any method known in the art. In many cases, the epitopes used for preparing variant-specific antibodies include at least 5, 6, 7, 8, 9, 10, 11, 12, or more amino acid residues that encompass or are selected from the unique region of the corresponding splice variant. In one example, an antigen is selected to include a sequence segment of amino acids 235-240 of GSK3-6b (SEQ ID NO:12), a sequence segment of amino acids 269-278 of GSK3-7b (SEQ ID NO:14), a sequence segment of amino acids 269-274 of GSK3Δ8 (SEQ ID NO:16), a sequence segment of amino acids 235-242 of GSK3Δ7-9 (SEQ ID NO:17), a sequence segment of amino acids 204-240 of GSK3Δ6-10 (SEQ ID NO:19), or a sequence segment of amino acids 76-82 of GSK3Δ2-11 (SEQ ID NO:20), where each sequence fragment includes at least five amino acid resides. In another instance, an antigen is selected to include a sequence segment of amino acids 230-240 of GSK3-6b, a sequence segment of amino acids 264-278 of GSK3-7b, a sequence segment of amino acids 264-279 of GSK3Δ8, a sequence segment of amino acids 230-247 of GSK3Δ7-9, a sequence segment of amino acids 199-240 of GSK3Δ6-10, or a sequence segment of amino acids 71-87 of GSK3Δ2-11, where each sequence fragment includes at least ten amino acid resides. In still another example, an antigen is selected to include a sequence segment of amino acids 225-240 of GSK3-6b, a sequence segment of amino acids 259-278 of GSK3-7b, a sequence segment of amino acids 259-284 of GSK3Δ8, a sequence segment of amino acids 225-252 of GSK3Δ7-9, a sequence segment of amino acids 194-240 of GSK3Δ6-10, or a sequence segment of amino acids 66-92 of GSK3Δ2-11, where each sequence fragment includes at least fifteen amino acid resides.

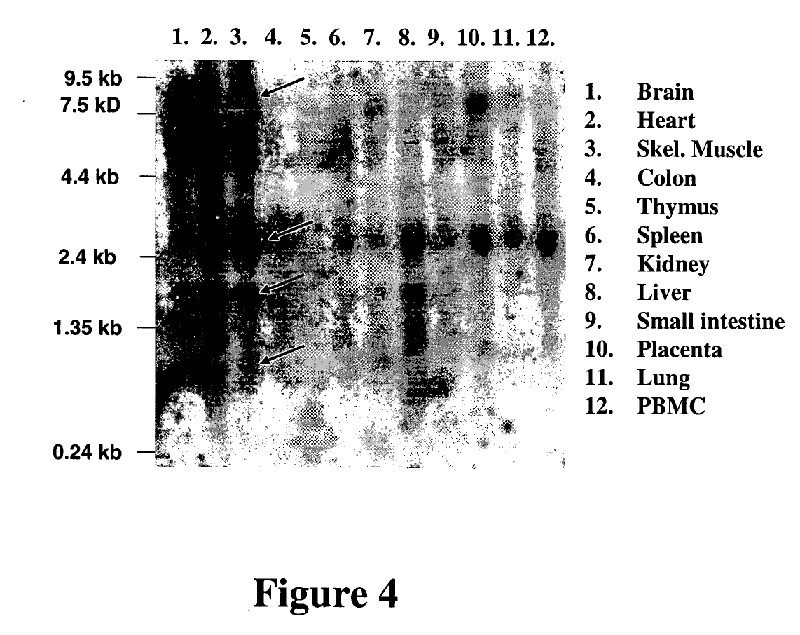

The suitability of each epitope/antigen thus selected can be evaluated based on its predicted 2D-structure and antigenic index. In one example, the 2D-structure prediction is made using the PSIPRED program (from David Jones, Brunel Bioinformatics Group, Dept. Biological Sciences, Brunel University, Uxbridge UB83PH, UK) (FIG. 4), and the antigenic index is calculated on the basis of the method described by Jameson and Wolf, (1988) CABIOS, 4:181-186. An antigenic index of 0.9 for a minimum of 5 consecutive amino acids is used as threshold for the program. Other peptides, scaffolds, antibody mimics, or high-affinity binders that can specifically interact with the GSK3β splice variants of the present invention can also be prepared.

Moreover, the present invention features polynucleotides comprising antisense sequences for the GSK3β splice variants of the present invention. An antisense molecule of the present invention can be complementary to a coding or non-coding region of a GSK3β splice variant. An antisense molecule can be complementary to the entire coding strand of a GSK3β splice variant or only a portion thereof. In many embodiments, the antisense molecules are oligonucleotides comprising, without limitation, about 10, 15, 20, 25, 30, 35, 40, 45, 50, or more nucleotide residues. The antisense molecules of the present invention can also comprise modified nucleotides to increase the biological stability of these molecules or the physical stability of the duplex formed between the antisense and sense polynucleotides.

In one embodiment, an antisense molecule of the present invention comprises a nucleotide sequence that is complementary to a unique region in the mRNA of a GSK3β splice variant of the present invention. Non-limiting examples of these unique regions include exon 6b (SEQ ID NO:21) for GSK3-6b, exon 7b (SEQ ID NO:22) for GSK3-7b, exon 8b (SEQ ID NO:23) for GSK3-8b, the junction region between exon 7 and exon 9 for GSK3Δ8 (e.g., nucleotides 806-820 of SEQ ID NO:4), the junction region between exon 6 and exon 10 for GSK3Δ7-9 (e.g., nucleotides 708-722 of SEQ ID NO:5), the junction region between exon 9 and exon 11 for GSK3Δ10 (e.g., nucleotides 1090-1104 of SEQ ID NO:6), the junction region between exon 6 and exon 10 for GSK3Δ6-10 (e.g., nucleotides 610-624 of SEQ ID NO:7), and the junction region between the remaining exon 2 and the remaining exon 11 for GSK3Δ2-11 (e.g., nucleotides 610-624 of SEQ ID NO:8).

In another embodiment, an antisense molecule of the present invention is designed to have at least 95%, 96%, 97%, 98%, 99% or 100% sequence homology to a unique region in the mRNA of a GSK3β splice variant of the present invention, but share no more than 90%, 80%, 70%, 60% or less sequence homology to the mRNAs encoding other GSK3β splice variants. In still another embodiment, an antisense molecule of the present invention is designed to be able to hybridize under stringent conditions to a unique region in the mRNA of a corresponding GSK3β splice variant, but not to the mRNAs encoding other GSK3β splice variants. The unique region in a GSK3β splice variant of the present invention can also be used to prepare probes or primers for the detection or amplification of that splice variant. In many instances, these probes or primers can hybridize under stringent conditions to the unique region from which they are derived, but not to the mRNAs or cDNAs that encode other GSK3β splice variants.

“Stringent conditions” are at least as stringent as a condition selected from Table 1. In Table 1, hybridization is carried out under the hybridization conditions (Hybridization Temperature and Buffer) for about four hours, followed by two 20-minute washes under the corresponding wash conditions (Wash Temp. and Buffer).

TABLE 1
Stringency Conditions

| Stringency | Poly-nucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying | | | | |
| # the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). | | | | |
| # For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165M). | | | | |

The present invention further contemplates the use of RNA interference (“RNAi”) to inhibit the expression of GSK3β splice variants in human cells or tissues. Any type of RNAi sequence can be used for the present invention. Non-limiting examples include short interfering RNA (siRNA) molecules or short hairpin RNA (shRNA). A variety of algorithms are available for RNAi sequence design. In one embodiment, the target sequences for siRNA include about 18, 19, 20 or more nucleotides. 2dT's can be added to the 3′ end during siRNA synthesis, creating an “AA” overhang. In many instances, the GC content of a target sequence is between 35% and 55%, and the sequence does not include any four consecutive A or T (i.e., AAAA or TTTT), three consecutive G or C (i.e., GGG or CCC), or seven “GC” in a row. More stringent criteria can also be employed. For instance, the GC content of a target sequence can be limited to between 45% and 55%, and any sequence having three consecutive identical bases (i.e., GGG, CCC, TTT, or AAA) or a palindrome sequence with 5 or more bases can be excluded. Furthermore, the target sequence can be selected to have low sequence homology to other variants or genes. The effectiveness of an RNAi molecule can be evaluated by introducing or expressing the RNAi sequence in a cell that expresses the GSK3β splice variant being targeted. A substantial change in the mRNA or protein level of the GSK3β splice variant is indicative of the effectiveness of the RNAi molecule in inhibiting the expression of that splice variant.

The RNAi molecules of the present invention can be designed to specifically inhibit a particular GSK3β splice variant. This can be achieved by selecting the RNAi target sequences from the unique region in the splice variant, such as exon 6b for GSK3-6b, exon 7b for GSK3-7b, or exon 8b for GSK3-8b. Examples of target sequences for siRNA are depicted in SEQ ID NOs:24-30 for GSK3-6b, SEQ ID NO:31 for GSK3-7b, and SEQ ID NOs:32-36 for GSK3-8b.

Furthermore, the present invention features isolated or purified GSK3β variants or polynucleotides encoding the same. An isolated protein (or polynucleotide) is substantially free from other proteins (or polynucleotides) or contains no more than an insignificant amount of contaminants that would interfere with its intended use. In many cases, a preparation of an isolated protein (or polynucleotide) contains less than 80%, 70%, 60%, 50%, 40%, 30%, 20%, 10%, 5%, or 1% by weight of other proteins (or polynucleotides). An isolated variant or a polynucleotide encoding the same can be prepared using any method known in the art, including but not limited to, standard recombinant DNA technology, standard protein or nucleic acid isolation techniques, or chemical synthesis.

In addition, the present invention features polypeptides that are functionally equivalent to the naturally-occurring GSK3β splice variants. These polypeptides share significant sequence homology to the naturally-occurring GSK3β splice variants and substantially retain the biological activities of the original splice variants (e.g., kinase activity or dominant-negative effect). In many embodiments, a functionally equivalent polypeptide of the present invention has at least 60%, 65%, 70%, 75%, 80%, 85%, 90%, 95%, 97%, 99%, or more global sequence identity or similarity to the original GSK3β splice variant, and retains at least 50%, 60%, 70%, 80%, 90% or more of the biological function (e.g., enhanced kinase activity or dominant-negative) of the original protein. Sequence identity or similarity can be determined using various methods known in the art, such as Basic Local Alignment Tool (BLAST), the algorithm of Needleman et al., (1970) J. Mol. Biol., 48:444-453, the algorithm of Meyers et al., (1988) Comput. Appl. Biosci., 4:11-17, or dot matrix analysis.

Additional polypeptide(s) can be fused to a variant of the present invention to facilitate its purification, detection, immobilization, folding, or targeting. The fused polypeptide(s) can also serve to increase the expression, solubility, or stability of the resulting protein. Other modifications, such as glycosylation, GPI anchor formation, or myristoylation, can also be introduced into a variant of the present invention.

A polynucleotide that encodes a variant of the present invention, or a functional equivalent thereof, can be DNA, RNA, or a modified form thereof. In many cases, the polynucleotides of the present invention are modified to increase their stabilities in vivo. Suitable modifications include, but are not limited to, the addition of flanking sequences at the 5′ or 3′ end, the use of phosphorothioate or 2-o-methyl instead of phosphodiesterase linkages in the backbone, and the inclusion of nontraditional bases such as inosine, queosine and wybutosine, as well as acetyl-, methyl-, thio- or other modified forms of adenine, cytidine, guanine, thymine and uridine.

In one embodiment, the polynucleotides of the present invention are expression vectors which comprise 5′ or 3′ untranslated regulatory sequences operatively linked to a sequence encoding a GSK3 variant or a GSK3 dominant-negative. The design of expression vectors depends on such factors as the choice of the host cells and the desired expression levels. Selection of promoters, enhancers, selectable markers, or other elements for an expression vector is a matter of routine design within the level of ordinary skill in the art. Many such elements are described in the literature and are available through commercial suppliers.

In another aspect, the present invention features pharmaceutical compositions comprising GSK3β modulators (including GSK3β dominant-negatives). The present invention also features pharmaceutical compositions comprising expression vectors or gene delivery vectors that encode GSK3β modulators (including GSK3β dominant-negatives). In one embodiment, a pharmaceutical composition of the present invention includes a polynucleotide comprising exon 6b, 7b or 8b (i.e., SEQ ID NO:21, 22 or 23, respectively). In another embodiment, a pharmaceutical composition of the present invention includes a polypeptide comprising an amino acid sequence encoded by exon 6b, 7b or 8b (i.e., amino acids 239-240 of SEQ ID NO:12, amino acids 273-278 of SEQ ID NO:15, and amino acids 304-316 of SEQ ID NO:16, respectively). The pharmaceutical compositions of the present invention can be formulated to be compatible with its intended route of administration. Non-limiting examples of routes of administration include parenteral, intravenous, intradermal, subcutaneous, oral, inhalation, transdermal, rectal, transmucosal, topical, and systemic administration. The administration can also be carried out by using implants.

A pharmaceutical composition of the present invention typically includes a pharmaceutically acceptable carrier and a therapeutically or prophylactically effective amount of a GSK3β modulator. A GSK3β modulator can be used either individually or in combination with other GSK3 modulators. The use of carrier media and agents for pharmaceutically active substances is well-known in the art. A pharmaceutical composition of the present invention can be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers. In one embodiment, the pharmaceutical compositions of the present invention are formulated and used as foams. Pharmaceutical foams include formulations such as emulsions, microemulsions, creams, jellies and liposomes. While basically similar in nature, these formulations vary in the components and the consistency of the final product. The preparation of such compositions and formulations is generally known to those skilled in the pharmaceutical and formulation arts and can be readily applied to the formulation of the compositions of the present invention. Dosage schedules for administration of a pharmaceutical composition of the present invention can be adjusted based on, for example, the affinity of the GSK3β modulator for its target, the half-life of the modulator, the site of pathology, the severity of the patient's condition, the patient's age, sex, and diet, the severity of any inflammation, and time of administration, as appreciated by those of ordinary skill in the art.

The present invention further features the use of GSK3β modulator for assessing the molecular bases of human diseases. As described above, many diseases involve dysfunction or dysregulation of GSK3β or its associated signaling pathways. Diseased cells or tissues can be isolated from a patient of interest and contacted with a GSK3β modulator of the present invention (e.g., a GSK3β dominant-negative, an antibody, an antisense, an siRNA, or an shRNA). A change in the disease phenotype of the cells/tissues in the presence of the GSK3 modulator, as compared to that in the absence of the modulator, is suggestive that GSK3β contributes to the disease condition in the patient of interest. Appropriate treatment(s) can therefore be selected for the patient of interest based on the molecular analysis of the disease.

It should be understood that the above-described embodiments and the following examples are given by way of illustration, not limitation. Various changes and modifications within the scope of the present invention will become apparent to those skilled in the art from the present description.

### EXAMPLES

### Example 1. Materials and Methods

Polymerase Chain Reaction

To identify novel isoforms of GSK3β in human bone, cDNA was made by the Superscript Plasmid System (Invitrogen, Carlsbad, Calif.) from 5 μg of total RNA which was isolated from a primary human bone cell line (Wyeth, Cambridge, Mass.) and from human liver poly-A RNA (Clontech, Palo Alto, Calif.). All RNA samples were treated with RQ1 RNase-free DNase according to the manufacturer's protocol (Promega, Madison, Wis.). The cDNA synthesis was primed by oligo(dT) and carried out with SuperScript II reverse transcriptase. Briefly, dNTPs (final concentration 0.7 mM) and 1 μg oligo(dT) primer were incubated at 65° C. for 5 min, then placed on ice for 2 min. Reverse transcription components were then added at final concentrations of: 1×RT buffer, 3.3 mM MgCl₂, 6.7 mM DTT, 40U RNAseOUT, and 1000U SuperScript II RT, then incubated at 37° C. for 5 min, then 48° C. for 1 hr.

PCR primers were designed to amplify the open-reading frame of GSK3β (RefSeq #NM_{—}002093). PCR was performed using Expand High Fidelity Kit (Roche Diagnostics, Mannheim, Germany) in 100 μL using final concentrations of: 1×reaction buffer, 0.8 mM dNTPs (Pekin Elmer, Boston, Mass.) 10 μM of each primer, 3.5U of Expand enzyme, and 100 ng of cDNA. The following primers were used to amplify GSK3-WT: Forward: 5′-CATATGTCGACATGTCAGGGCGGCCCAGAA-3′ (SEQ ID NO:37), Reverse: 5′-GATATGCGGCCGCTCAGGTGGAGTTGGAAGCTG-3′ (SEQ ID NO:38). Amplification parameters were: 96° C. 2 min, 40 cycles of 96° C. 30 sec, 60° C. 45 sec, 72° C. 1 min 30 sec, and 72° C. 7 min. PCR products were purified by QIAquick columns (Qiagen, Valencia, Calif.), digested by SalI/NotI restriction enzymes (NE Biolabs, Beverly, Mass.) and cloned into pBluescript SK- (Stratagene, La Jolla, Calif.) with T4 DNA ligase (NE Biolabs).

Isolation of Variants

Individual clones were prepared by QIAprep 96 Turbo Miniprep kits (Qiagen), restriction digested by SalI/NotI, and visualized by 1% agarose gel electrophoresis with ethidium bromide. Clones were sorted by insert sizes and processed for full-length sequence analysis. Clones that were identified as variants to GSK3β were analyzed further to validate the potential intron/exon boundaries based on the available public genomic sequence data. Clones with novel insertions were analyzed to see if those sequences could be found within the corresponding intragenic regions.

Bioinformatic Analysis

To determine if the novel inserts (exons) 6b, 7b and 8b were artifacts of cDNA synthesis or transcription, a search was initiated to locate these sequences within the appropriate intragenic regions of GSK3β. 8b was previously confirmed to be located between exons 8 and 9 (Schaffer et al., (2003) Gene, 302:73-81). GSK3β is located at chromosomal position 3q13.3 and the reference genomic sequence was obtained from NCBI (accession #NT_{—}029257). Sequence alignments were created using Sequencher 4.1 software. The base positions of each alignment of 6b and 7b to the genomic sequence were recorded and compared to the genomic locations of all GSK3β exons 1-11. For the novel clones with deletions, an analysis was done to measure how closely the deleted regions follow the GT-AG rule (by consensus sequence obtained from Schaffer et al., (2003) Gene, 302:73-81) for intron splicing.

Northern Blots

For clones with nucleotide insertions of <60 bp between the known exon boundaries of GSK3β, antisense oligonucleotides were designed to the novel sequence (Integrated DNA Technologies, Coralville, Iowa). Oligonucleotides were end-labeled with γ-P³² labeled dCTP (Perkin Elmer) using T4 polynucleotide kinase (Stratagene). Reaction conditions in 25 μL were: 1×buffer, 35 μCi γ-P³² dCTP, 25 pmol oligo, and 1 μL T4 kinase. Unincorporated nucleotides were removed with NICK columns (Amersham, Piscataway, N.J.). For GSK3β-WT (SEQ ID NO:1) and clones with nucleotide insertions >60 bp, PCR primers were ordered to amplify these regions (Integrated DNA Technologies, Coralville, Iowa). PCR products were radio-labeled with α-P³² labeled dCTP using the Prime-It II random primer kit (Stratagene). Reaction conditions in 50 μL were: 1×buffer, 50 μCi α-P³² dCTP, 100 ng DNA, 10 μL random primers, and 1 μL Klenow (NE Biolabs). These were also purified by NICK columns as above. The amount of incorporated radio-nucleotide was measured with a scintillation counter. Total counts for each probe were as follows: GSK3-WT=7.5×10⁶ cpm, GSK3-6b=9.2×10⁶ cpm, GSK3-7b=6.9×10⁶ cpm, GSK3-8b=1.3×10⁷ cpm. Multiple tissue northern blots with mRNA from 12 different human tissues were probed (Clontech). Hybridization was carried out in 10 ml QuickHyb solution (Strategene) for 1 hour at 37° C. for oligonucleotide probes and 65° C. for cDNA probes. Blots were washed twice in 2×SSC-0.1% SDS for 20 minutes. The radioactive bands were visualized by autoradiotography using BioMax MS X-ray film (Kodak, Rochester, N.Y.).

Expression in Bone Cell Lines

Human bone cell lines U2OS, SAOS-2, MG-63 (osteosarcomas) and TE-85 (osteoblastic) were grown to confluency and harvested in lysis buffer by RNeasy Midi-prep kit (Qiagen). RNA was quantitated by spectrophotometer, treated with DNase as previously described, and 5 μg were converted to cDNA using the First-Strand cDNA Synthesis kit (Invitrogen). The cDNA synthesis was carried out as previously described. The cDNA was diluted to 100 ng/μL for use as a template in PCR amplification. For PCR amplification of the novel insertions, the conditions were also the same as described previously. Oligonucleotides were designed to amplify only the novel regions of the GSK3β variants with insertions. Primers for “6b”: Forward: 5′-CAGAGTTGATCTTTGGAGCCACTGA-3′ (SEQ ID NO:39), Reverse: 5′-AGACCATACATCAGTACGGAAGGAG-3′ (SEQ ID NO:40). Primers for “7b”: Forward: 5′-GCTGAGCTGTTACTAGGACAACCAA-3′ (SEQ ID NO:41), Reverse: 5′-GTTCCCAGGACAGCTT TCCCAGG-3′ (SEQ ID NO:42). Primers for “8b”: Forward: 5′-CAGAGAAATGAACCCAA ACTACACAG-3′ (SEQ ID NO:43), Reverse: 5′-GGTCGGAAGACCCGCACTCCTGA-3′ (SEQ ID NO:44) (Integrated DNA Technologies). PCR products were visualized by gel electrophoresis. To confirm that the correct products were amplified, PCR bands of expected sizes were cloned by TOPO TA kit into pCRII-TOPO according to the manufacturer's protocol (Invitrogen). The cloned PCR fragments were then confirmed by DNA sequence analysis.

Subcloning

The GSK3β clones in pBluescript were subcloned into the mammalian expression vector pcDNA3.1 (Invitrogen). This was done by utilizing the restriction sites SalI and NotI. The inserts were separated by gel electrophoresis and cleaned by Nucleospin columns (Clontech). About 100 ng of each purified insert was ligated to SalI and NotI cut pCDNA3.1 vector with 400 U of T4 ligase (NE Biolabs) in 1×buffer. Subclones were confirmed by gel electrophoresis and full-length sequence analysis.

Cell Culture

U2OS cells (American Tissue Type Culture, USA) were maintained in McCoy's 5a medium supplemented with 10% FBS+1% L-glutamine+1% penicillin/streptomycin. The luciferase assay medium (McCoy's 5a medium) was supplemented with 1% FBS+1% L-glutamine+1% penicillin/streptomycin. Cells were incubated at 37° C., 5% CO₂. Cells were seeded in white-walled 96-well tissue culture plates (Corning #3903, VWR International West Chester, Pa.) with 10,000 cells seeded per well. SAOS2 cells were also maintained in McCoy's 5a medium while MG63 and TE85 were maintained in DMEM medium supplemented with 10% FBS+1% L-glutamine+1% penicillin/streptomycin.

TCF-Luciferase Reporter Assay

Transfection conditions for the U2OS cells were modified, using a final concentration 0.5% Lipofectamine 2000 (Invitrogen) in 100 μL final volume (96 well format). The reporter plasmids used were TOPFLASH TCF (Upstate Biotechnology, Lake Placid, N.Y.) and Renilla pRL-SV40 (Promega). Final concentrations of the plasmids were: 4 ng/μL of TOPFLASH, 25 pg/μL Renilla and 1 ng/μL of each GSK3β construct. All were diluted in Opti-Mem medium (Invitrogen) in a final volume of 100 μl. Transfections were performed at 37° C. for 4 hr. After this incubation, the medium was replaced with McCoy's 5a medium with 1% FBS+1% L-glutamine with no antibiotics added. To activate Wnt signaling, conditioned medium (CM) from stable L-cells expressing Wnt3a (ATCC) was used. A 1:4 dilution of Wnt3a CM in McCoy's 5a medium was used to treat the cells for 24 hrs. Untreated cells received 1:4 dilution of CM from L-cells as a control. Following Wnt3a treatment, the cells were washed once in PBS and harvested in 1×lysis buffer (Promega).

Reporter gene activity was measured with a luminometer using the Dual Luciferase Assay System for Firefly and Renilla (Promega). With this system, luciferase activity was normalized for transfection efficiency by measuring Renilla activity. Each luciferase reading was normalized to Renilla and expressed as a Luciferase/Renilla ratio. Ratios for the GSK3β variants were compared to wild-type GSK3P, and converted to fold change either above or below this positive control. Statistical significance of changes among the variants were measured using the Student's t-test and were considered significant at p-values<0.05.

### Example 2. Identification of Novel Variants of GSK3

A PCR-based approach was designed to search for novel sequence variants of GSK3β in a primary bone cell line (non-immortalized). Primers were designed to amplify the wild-type (GSK3-WT) open-reading frame (ORF), and the products of this PCR amplification (the majority being the GSK3-WT 1260 bp band) were subcloned. Subclones were selected based on differences in size from the endogenous GSK3-WT by gel electrophoresis. After DNA sequencing, divergent regions from the GSK3-WT were identified. A total of 8 independent clones were discovered that were either missing portions of the GSK3β ORF or had novel sequences inserted between exons within the coding region.

Three subclones were identified with insertions between exons (FIGS. 1B, 1C and 1D). A genome contig (NT_{—}029257) was identified to contain exons 6 through 9 of GSK3β. The novel regions of these 3 clones were mapped to this public genomic DNA sequence data from chromosome 3, indicating that these clones were actual novel splice variants and not just artifacts of transcription or of cDNA generation. All three insertions were found to be located between the appropriate exons within the genomic sequence data, suggesting the insertions were not spurious DNA integration. A search of the GSK3β pseudogene on chromosome 11 for the insertions came up negative, which suggests the insertions are not derived from a pseudogene.

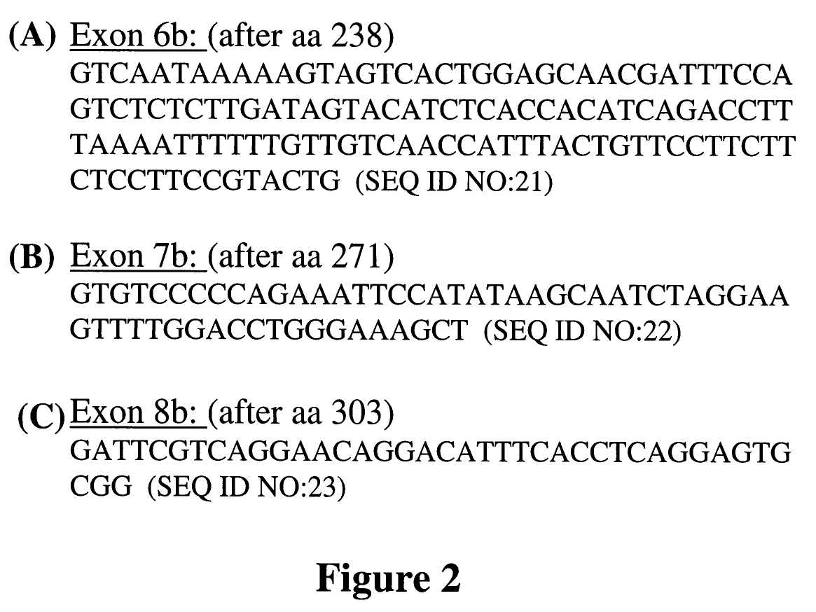

A clone with a novel 125 bp insertion was found between exons 6 and 7 (FIGS. 1B and 2A, and SEQ ID NO:1). This insertion is predicted to cause a shift in the GSK3β reading frame and introduce a stop codon at aa 241. This insertion creates a truncated ORF of 723 bp (GSK3-6b and SEQ ID NO:11) and encodes a protein having the amino acid sequence of SEQ ID NO:12). A second clone was found with a novel 56 bp insertion between exons 7 and 8 (FIGS. 1C and 2B, and SEQ ID NO:2). This insertion also results in a frame shift and introduces a stop codon at aa 279, creating a truncated clone with an 837 bp ORF (GSK3-7b and SEQ ID NO:13) which encodes a protein having the amino acid sequence of SEQ ID NO:14. A third clone (GSK3-8b) was identified with a 39 bp insertion between exons 8 and 9. This clone was also isolated in the brain and the insertion was reported as “exon 8b” (Schaffer et al., (2003) Gene, 302:73-81; and Mukai et al., (2002) J. Neurochemistry, 81:1073-1083). The insertion maintains the original ORF for GSK3β, but adds 13 additional amino acids after aa 303 (FIGS. 1D and 2C). This clone is referred to as GSK3-8b (SEQ ID NO:3) and encodes a protein having the amino acid sequence of SEQ ID NO:15.

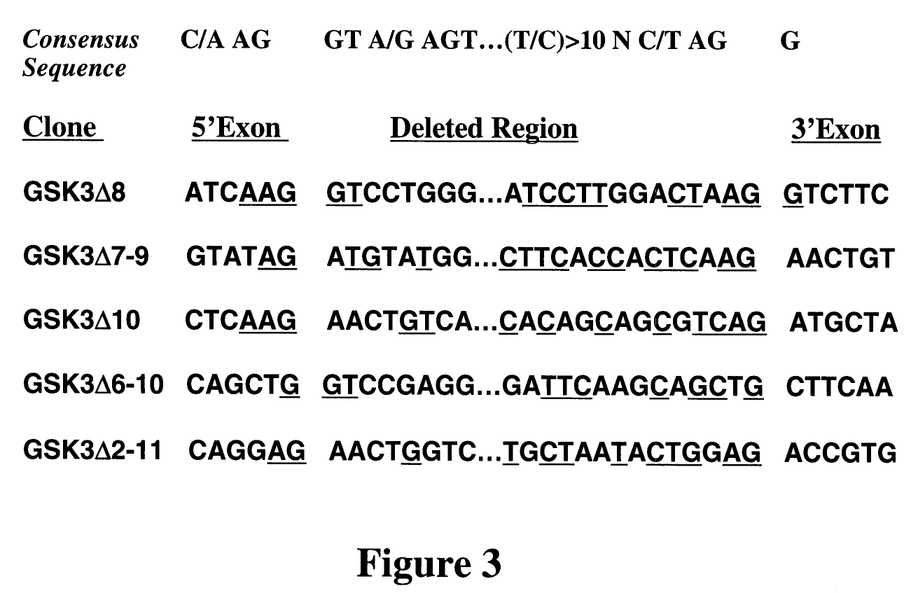

Five GSK3β variants were found with whole or partial exon deletions (FIGS. 1E, 1F, 1G, 1H, and 1I). These clones were further analyzed to determine whether each GSK3β variant maintains an open-reading frame. Four of these clones GSK3Δ8 (SEQ ID NO:4), GSK3Δ7-9 (SEQ ID NO:5), GSK3Δ10 (SEQ ID NO:6), and GSK3Δ2-11 (SEQ ID NO:8) retain the original open-reading frames despite the missing regions, and each clone encodes a protein having the amino acid sequence of SEQ ID NO:16, SEQ ID NO:17, SEQ ID NO:18, or SEQ ID NO:20, respectively. GSK3Δ10 was also described by Schaffer et al., (2003) Gene, 302:73-81. One clone, GSK3Δ6-10 had a 541 bp deletion causing a shift in the opening reading frame that can extend the length of the expression product of the gene. A partial sequence of GSK3Δ6-10 is depicted in SEQ ID NO:7 which encodes a protein having the amino acid sequence of SEQ ID NO:19. These clones were also analyzed to measure how closely the deleted regions follow the GT-AG rule (by consensus sequence described in Schaffer et al., (2003) Gene, 302:73-81) for intron splicing (FIG. 3). All five variants with deletions had similarities to the consensus sequence. GSK3Δ8 (15) and GSK3Δ7-9 (16) had the most base matches to the consensus sequence. GSK3Δ10, GSK3Δ6-10, and GSK3Δ2-11 had 13, 10 and 12 matches respectively. These splice junction similarities suggest the probability that these sequences were recognized and processed by the spliceosome, is more than a chance occurrence.

### Example 3. Northern Blot and RT-PCR Analyses

The unique regions in the variants with insertions were used to design probes specific to each corresponding variant. Northern blots were performed to validate these insertion variants. Tissue expression data for each variant were also obtained. The Northerns consisted of an array of mRNA from 12 different human tissues pre-normalized for the amount of RNA loaded per lane. Radio-labeled probes used to detect GSK3-WT and GSK3-7b were generated by PCR. Probes for GSK3-6b and GSK3-8b were created from end-labeled oligonucleotides. The probes used for GSK3-6b, 7b, and 8b were designed to only detect the novel insertions (exons).

Northern results for GSK3-WT (FIG. 4) mirrored the published data for GSK3β where two bands of 8.3 kb and 2.8 kb were reported (Lau et al., (1999) J. Peptide Res., 54:85-91). The large size differences of these bands from the expected 1263 bp are likely due to multiple polyadenylation sites on the 3′ UTR region of GSK3β. GSK3-WT was detectable in most arrayed tissues. At least two additional bands around 1.0 kb and 2.0 kb were observed in FIG. 4, suggesting that these bands are novel variants of GSK3β.

Northern results for GSK3-8b (FIG. 5) were similar to that of GSK3-WT. Two major bands of about 4.5 kb and about 2.2 kb were observed in most tissues. There are two additional bands of about 1.0 kb and about 0.5 kb that are relatively strong in heart, skeletal muscle and liver. These bands may represent additional variants that contain exon 8b.

GSK3-6b and GSK3-7b Northern results revealed tissue specific expression patterns upon the overnight exposure to the film. One band for GSK3-6b was observed at about 1.8 kb in skeletal muscle (FIG. 6). GSK3-7b was observed in heart, skeletal muscle, kidney, liver, small intestine and placenta, and had multiple bands in many of these tissues. For instance, bands of about 1.6 kb and about 1.4 kb, and a much larger band of about 8.0 kb, were visible in skeletal muscle (FIG. 7).

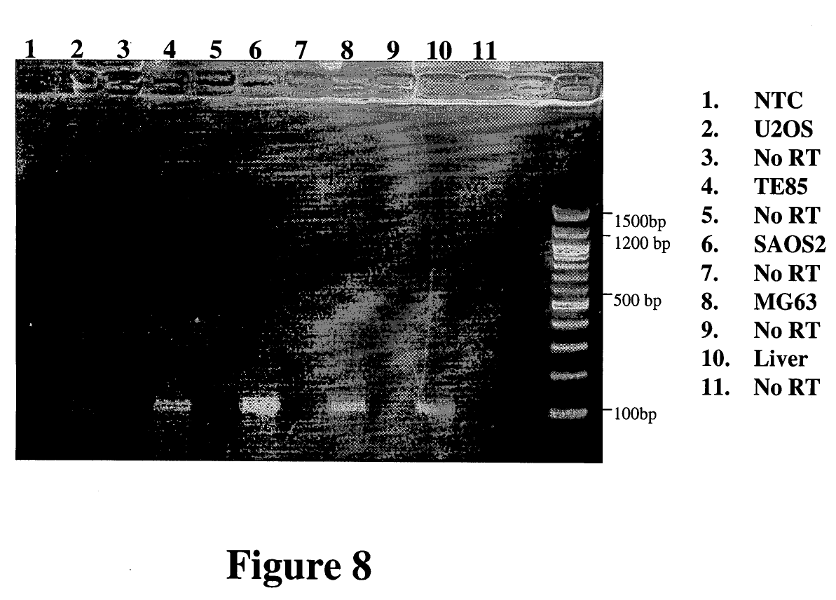

To confirm the expression of the novel variants in bone, total RNA was harvested from several different human bone cell lines; osteosarcoma cells (U2OS, SAOS-2, MG-63) and osteoblastic cells (TE-85). Liver poly-A mRNA was used as a positive control for GSK3-WT, and was detectable in all samples. Primers were designed to amplify the insertion (exon) sequences of GSK3-6b, GSK3-7b, or GSK3-8b. GAPDH was used as a normalization control gene (FIG. 8). The “No-RT” controls, run to control for amplification of contaminating genomic DNA, were consistently clean.

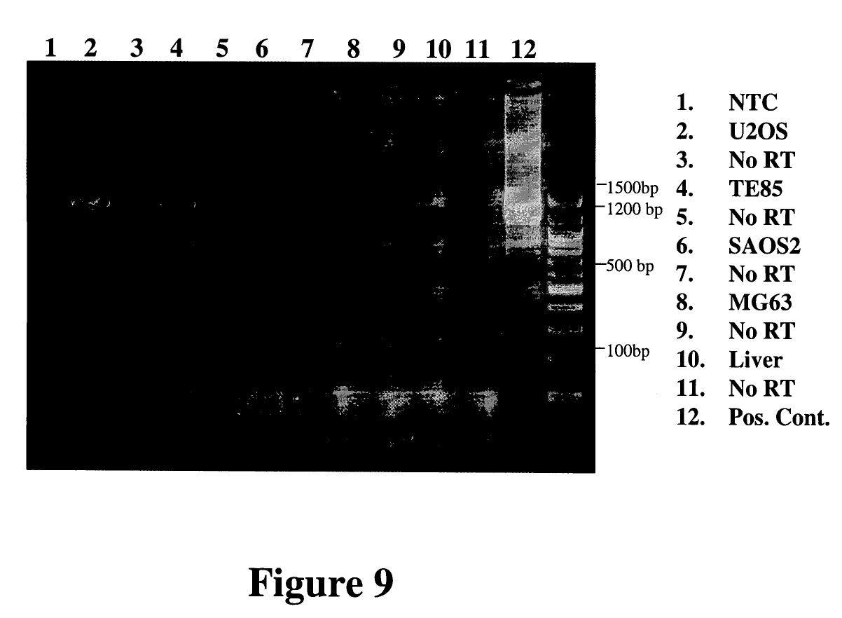

GSK3-WT was detected in all the bone cell lines except MG63 (FIG. 9). This reconfirms the original experiment demonstrating wild-type GSK3β exists in bone. There was also an additional band of about 300 bp detected in U2OS cells (lane 2). Its size suggests it may be GSK3Δ2-11. Exon 6b was expressed in U2OS, TE85 and liver (the band marked by arrow in FIG. 10A). Exon 7b was expressed in U2OS, SAOS2, and MG63, but not liver (the band marked by arrow in FIG. 10B). Exon 8b was expressed in all bone cell lines and in liver (the band marked by arrow in FIG. 10C). For each exon, a representative PCR band from a bone cell line was subcloned and sequenced, proving that the correct product was amplified. These results suggest all of three variants (GSK3-6b, GSK3-7b, and GSK3-8b) are expressed in at least one bone cell line.

### Example 4. TCF Luciferase Assay

A Wnt-responsive TCF-luciferase reporter gene assay was set up to measure each GSK3β variant's activity compared to the wild-type GSK3β in canonical Wnt signaling. The TCF-luciferase reporter gene assay is a common method for studying gene expression and cell physiology of the Wnt pathway. Activation of the Wnt pathway leads to stabilization of β-catenin and transcription of TCF target genes, such as luciferase whose expression can be monitored by luminescence.

This assay used a dual-luciferase system. Co-transfections were performed with the reporter plasmid TOPFLASH, containing TCF binding sites upstream of the luciferase open-reading frame. The second reporter plasmid used was pRL-SV40 containing the cDNA encoding Renilla luciferase. Expression of Renilla is under the control of the SV40 early promoter, which provides strong expression of Renilla in a variety of cell types. Renilla activity is measured and used as a normalization tool in order to control for cell transfection efficiency.

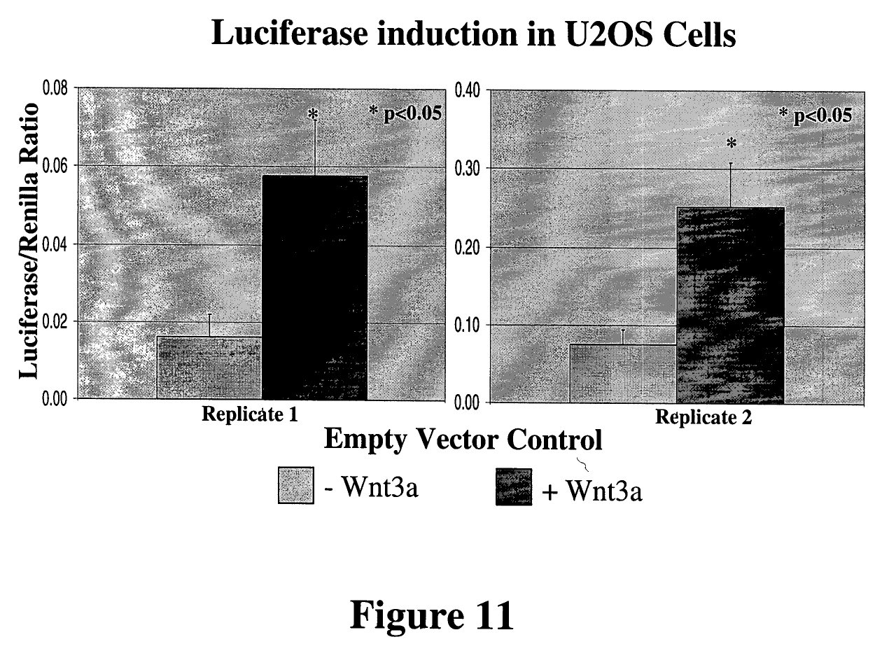

The assay was performed in a readily transfectable bone cell line, U2OS. The GSK3β variants were subcloned into the expression vector pCDNA3.1. A concern with this experiment was that the basal levels of Wnt pathway activation in the U2OS cells may be low for detection or for measurement of changes. Therefore, Wnt3a was used to activate the Wnt pathway to increase the window where changes caused by GSK3β or the variants could be measured. Conditioned medium from a stable L-cell line expressing Wnt3a was used to activate the Wnt pathway in this system. Conditioned medium from normal L-cells was used as the control in this experiment and is referred to as “−Wnt3a” (FIG. 11). Two replicate experiments were run on different days. Activation of the Wnt pathway by Wnt3a did in fact lead to in changes in luciferase levels. A consistent (p<0.05) 3-fold activation of luciferase was observed over cells that were untreated. FIG. 11 shows this repeatable 3-fold activation of the Wnt pathway by Wnt3a in replicate experiments.

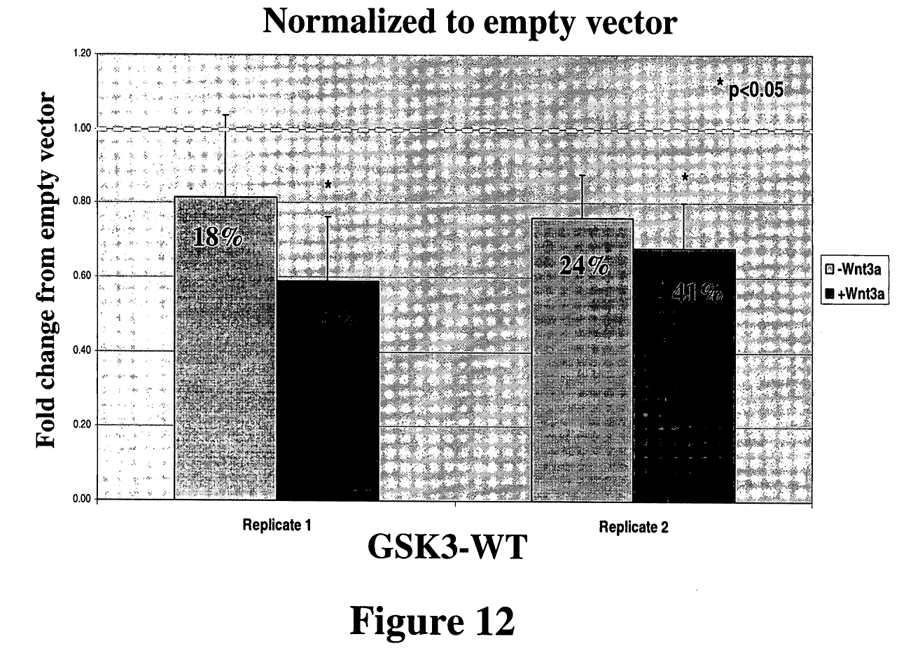

It was expected that the addition of wild-type GSK3β would increase the phosphorylation of β-catenin, which would then be targeted for degradation to the proteosome. This would lead to less transcription of TCF target genes and would result in reduced luminescence from the reporter construct. This in fact was observed when GSK3-WT was transfected into the U2OS as compared to empty vector (FIG. 12). An 18% and a 24% (p<0.05) inhibition of the Wnt pathway was measured in cells that were untreated with Wnt3a in replicate experiments. Cells that were treated with Wnt3a showed about a 32% and a 40% (p<0.05) inhibition, suggesting that the Wnt3a helps increase the window in which changes in inhibition can be detected. The inhibition of the Wnt pathway by GSK3-WT was used as a baseline to which GSK3β variants were compared.

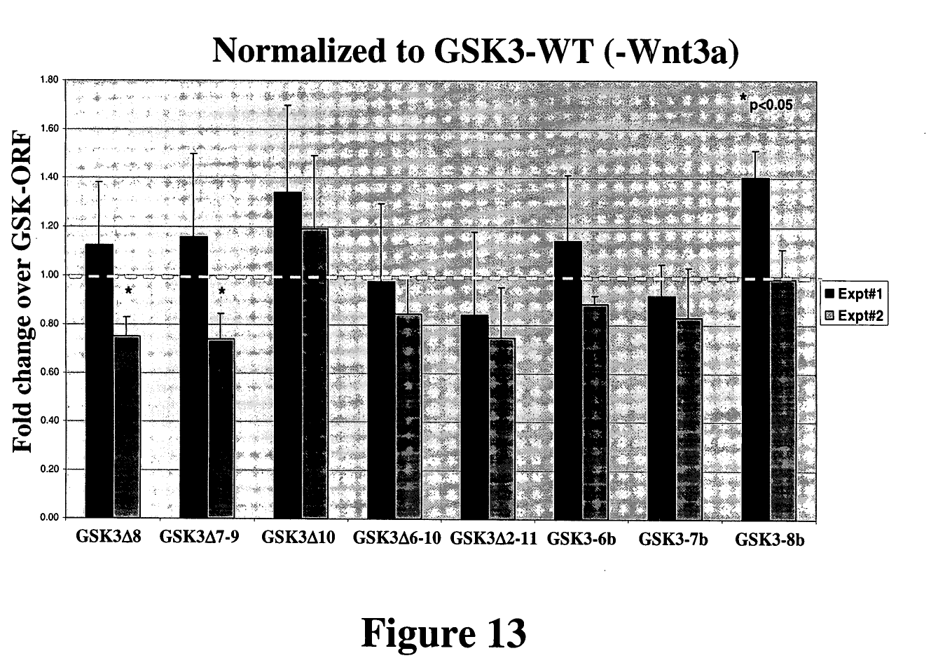

GSK3β variants were normalized to GSK3-WT in order to emphasize the differences in activity due to their insertions or deletions. With the exception of a single replicate experiment for GSK3Δ8 and GSK3Δ7-9, transfection of the variants into the U2OS cells that were not treated with Wnt3a showed insignificant changes in the Wnt pathway activity compared to GSK3-WT (FIG. 13). When the cells were treated with Wnt3a, however, different variants exhibited different effects on the activation of the Wnt pathway.

There appears to be three categories in which the variants can be classified based on their activities in this assay. The first category is made up of variants that have activities equal to or better than GSK3-WT. This includes GSK3Δ8 and GSK3-8b. GSK3Δ8 had nearly identical activity as GSK3-WT, while GSK3-8b seemed to inhibit the Wnt pathway 30% and 48% greater in replicate experiments. This suggests that exon 8b is involved in preventing GSK3β inactivation, which is believed to occur through the binding of FRAT to GSK3.

A second category contains the variants that are as effective as or less effective than GSK3-WT in inhibiting the Wnt pathway (no significant differences). GSK3Δ7-9 and GSK3Δ10 had small increases in luciferase activity. In particular, GSK3Δ7-9 had about 1.4-1.5 fold increases in luciferase activity, while GSK3Δ10 had about 1.5-1.9 fold increases and was significant in one experiment.

A third category consists of variants that had significant increases in luciferase activity over GSK3-WT (p<0.05). The variants with increases in luciferase activity were: GSK3Δ6-10 (about 2.0-2.1 fold), GSK3Δ2-11 (about 1.9-2.9 fold), GSK3-6b (about 2.0-2.8 fold), and GSK3-7b (about 2.3-3.0 fold). The differences were consistent in both replicate experiments for each of variants in this category. The activation of the Wnt pathway by these variants indicates that they are acting as dominant-negatives in which they out-compete the endogenous levels of GSK3β to reverse its inhibition on the Wnt pathway.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. An isolated protein comprising:
(a) a human glycogen synthase kinase 3β (GSK3β) amino acid sequence in which five or more consecutive amino acids encoded by a GSK3-WT exon are absent, wherein the GSK3-WT exon is selected from the group consisting of exon 2, exon 3, exon 4, exon 5, exon 6, exon 7, exon 8, exon 9, and exon 11; or
(b) an amino acid sequence at least 90% identical to (a).

2. The isolated protein of claim 1, wherein the isolated protein comprises an amino acid sequence selected from the group consisting of SEQ ID NOs: 12, 14, 16, 17, 19 and 20.

3. The isolated protein of claim 1, wherein the GSK3-WT exon is selected from the group consisting of exon 7, exon 8, and exon 9 and wherein five or more consecutive amino acids encoded by GSK3-WT exon 10 are also absent.

4. The isolated protein of claim 3, wherein the isolated protein is selected from the group consisting of SEQ ID NOs: 12, 14, 19 and 20.

5. A method of inhibiting an activity of glycogen synthase kinase 3β (GSK3β) in human cells, said method comprising introducing the isolated protein of claim 3 into said cells such that the activity of GSK3β in said cells is inhibited.

6. The method of claim 5, wherein said human cells are bone cells, neuronal cells, adipose tissue cells, or metabolic tissue cells.

7. A pharmaceutical composition comprising the isolated protein of claim 3.

8. An antibody capable of binding to the amino acid sequence in the isolated protein of claim 2, but not to the amino acid sequence depicted in SEQ ID NO:10.

9. An isolated polynucleotide comprising an open reading frame encoding the isolated protein of claim 1.

10. An isolated polynucleotide capable of inhibiting the expression of a GSK3β splice variant by RNA interference or an antisense mechanism, wherein said isolated polynucleotide comprises a sequence which is complementary or antisense to at least a portion of an mRNA encoding said amino acid sequence in the isolated protein of claim 2, but not to mRNAs encoding GSK3-WT.

11. A method of identifying agents capable of modulating a biological activity of the isolated protein of claim 2, said method comprising detecting said biological activity in the presence or absence of a candidate molecule, wherein a change in said biological activity in the presence of said candidate molecule as compared to that in the absence of said candidate molecule is indicative that said candidate molecule is capable of modulating said biological activity.

12. An agent identified by the method of claim 11, wherein said agent is capable of inhibiting a dominant-negative effect of the isolated protein on the activity of a full-length GSK3β in Wnt signaling, and wherein the isolated protein comprises an amino acid sequence selected from the group consisting of SEQ ID NOs: 12, 14, 19 and 20.

13. A method for inhibiting GSK3β activity in human cells, said method comprising introducing into said cells an agent selected from the group consisting of:
an antibody capable of binding to a protein encoded by a GSK3β splice variant, said GSK3β splice variant being selected from the group consisting of GSK3Δ8 and GSK3Δ7-9, and said antibody being incapable of binding to a full-length GSK3-WT protein;
an antisense molecule for said GSK3β splice variant, but not for GSK3-WT;
an expression vector encoding said antisense molecule;
an RNAi molecule for said GSK3β splice variant, but not for GSK3-WT;
one or more expression vectors encoding said RNAi molecule; and
an expression vector comprising an open reading frame encoding the protein of claim 3.

14. The method of claim 13, wherein said human cells are bone cells, neuronal cells, adipose tissue cells, or metabolic tissue cells.

15. A method for inhibiting GSK3β activity in human bone cells, said method comprising introducing into said cells an agent selected from the group consisting of:
an antibody capable of binding to a protein encoded by a GSK3β splice variant, said GSK3β splice variant being selected from the group consisting of GSK3-8b and GSK3Δ10, and said antibody being incapable of binding to a GSK3-WT protein;
an antisense molecule for said GSK3β splice variant, but not for GSK3-WT;
an expression vector encoding said antisense molecule;
an RNAi molecule for said GSK3β splice variant, but not for GSK3-WT; and
one or more expression vectors encoding said RNAi molecule.

16. A method for treating a disease that is responsive to modulation of a GSK3β-associated signaling pathway, said method comprising administering a pharmaceutical composition to a patient who has said disease, said pharmaceutical composition comprising an agent selected from the group consisting of:
an antibody capable of binding to a protein encoded by a GSK3β splice variant, said GSK3β splice variant being selected from the group consisting of GSK3Δ8 and GSK3Δ7-9, and said antibody being incapable of binding to a GSK3-WT protein;
an antisense molecule for said GSK3β splice variant, but not for GSK3-WT;
an expression vector encoding said antisense molecule;
an RNAi molecule for said GSK3β splice variant, but not for GSK3-WT;
one or more expression vectors encoding said RNAi molecule;
a polypeptide comprising an amino acid sequence selected from the group consisting of SEQ ID NOs: 12, 14, 19 and 20; and
an expression vector comprising an open reading frame encoding said polypeptide.

17. The method of claim 16, wherein said disease is a bone loss disease, a neurodegenerative disease, or a metabolic disease.

18. The method of claim 16, wherein said disease is osteoporosis, Type 2 diabetes, obesity or Alzheimer's disease.

19. A method of treating a disease characterized by bone loss, said method comprising introducing a pharmaceutical composition into bone cells of a patient who has said disease, wherein said pharmaceutical composition comprises an agent selected from the group consisting of:
an antibody capable of binding to a protein encoded by a GSK3β splice variant, said splice variant being selected from the group consisting of GSK3-8b and GSK3Δ10, and said antibody being incapable of binding to a GSK3-WT protein;
an antisense molecule for said splice variant, but not for GSK3-WT;
an expression vector encoding said antisense molecule;
an RNAi molecule for said splice variant, but not for GSK3-WT; and
one or more expression vectors encoding said RNAi molecule.

20. A method of identifying agents capable of inhibiting a biological activity of GSK3-8b, said method comprising detecting said biological activity in the presence or absence of a candidate molecule, wherein a reduction in said biological activity in the presence of said candidate molecule as compared to in the absence of said candidate molecule is indicative that said candidate molecule is an inhibitor of GSK3-8b.

21. An isolated protein comprising an amino acid sequence which includes:
a sequence segment of amino acids 235-240 of GSK3-6b;
a sequence segment of amino acids 269-278 of GSK3-7b;
a sequence segment of amino acids 269-274 of GSK3Δ8;
a sequence segment of amino acids 235-242 of GSK3Δ7-9;
a sequence segment of amino acids 204-240 of GSK3Δ6-10; or
a sequence segment of amino acids 76-82 of GSK3Δ2-11,
wherein each said sequence segment comprises at least five amino acid residues.

22. An isolated polynucleotide comprising an open reading frame encoding said isolated protein of claim 21.
